(19) United States
(12) Patent Application Publication
(10) Pub. No.: US 2023/0286934 A1
(43) Pub. Date: Sep. 14, 2023

(54) Use of Multi-Kinase Inhibitors to Treat RNA Virus Infections

(71) Applicant: Icahn School of Medicine at Mount Sinai, New York, NY (US)

(72) Inventors: Premkumar Reddy, New York, NY (US); Adolfo Garcia-Sastre, New York, NY (US); Ramana M.V. Reddy, New York, NY (US); Kris White, New York, NY (US)

(21) Appl. No.: 18/008,282

(22) PCT Filed: Jun. 8, 2021
(86) PCT No.: PCT/US2021/036407

§ 371 Date: Dec. 5, 2022

Related U.S. Application Data
(60) Provisional application No. 63/036,243, filed on Jun. 8, 2020.

(51) Int. Cl. C07D 279/16; A61P 31/14

(52) U.S. Cl. CPC C07D 279/16; A61P 31/14

(57) ABSTRACT

Provided herein are methods for treating a virus (e.g,. an RNA virus, such as a SARS-CoV-2) infection or a disease associated therewith (e.g., COVID-19) comprising administering a compound of Formula (I), or a salt thereof, or a composition thereof to a subject (e.g., a human subject). Also provided herein are methods for treating a virus (e.g,. an RNA virus, such as a SARS-CoV-2) infection or a disease associated therewith (e.g., COVID-19) comprising administering 108110 or a composition thereof to a subject (e.g., a human subject). In addition, provided herein are methods for treating a virus (e.g., an RNA virus, such as a SARS-CoV-2) infection or a disease associated therewith (e.g., COVID-19) comprising administering 108600 or a composition thereof to a subject (e.g., a human subject).

### FIELD OF THE INVENTION

The present invention is directed to methods for treating a virus (e.g,. an RNA virus, such as a SARS-CoV-2) infection or a disease associated therewith (e.g., COVID-19) by administering a compound of Formula (I), or a salt thereof, or a composition thereof to a subject. Also provided herein are methods for treating a virus (e.g,. an RNA virus, such as a SARS-CoV-2) infection or a disease associated therewith (e.g., COVID-19) by administering compound 108110, compound 108600 or a composition thereof to a subject (e.g., a human subject).

### 1. Background

There is an urgent need to develop therapeutics to treat COVID-19 and diagnostics to detect severe acute respiratory syndrome coronavirus-2 (SARS-CoV-2). As of Jun. 1, 2020, more than 6,226,409 people globally have tested positive for SARS-CoV-2. In addition, as of Jun. 1, 2020, globally more than 373,883 people have died from COVID-19. Currently, there is no approved vaccine or therapeutic to prevent or treat COVID-19.

108600 and 108110 are synthetic 2-benzyliden-2H and 2-besylidene derivatives, previously developed as anti-cancer agents. However, there have been no reported uses associated with RNA viruses, such as coronaviruses.

### 2. Summary

In one aspect, provided herein is the use of a compound described herein as an antiviral (e.g., an antiviral for RNA virus infections). The use of a compound described herein as an antiviral is based, in part, on the discovery that it inhibits the replication of SARS-CoV-2, a positive-sense single-stranded RNA virus. See Section 5, infra. RNA viruses utilize cellular signaling machinery for their replication and virus assembly, suggesting that inhibition of one or more of these signaling pathways could result in the inhibition of viral replication. For example, SARS-CoV-2 infection is initiated by the interaction of viral spike protein with host cell surface receptors such as Angiotensin-converting enzyme 2 (ACE2) or CD147. This event results in the fusion of cellular and viral membranes leading to the release of viral genome into the cellular cytoplasm. Infection of lung epithelial cells with SARS-CoV-2 induces the activation of multiple cellular signaling pathways leading to enhanced replication of the virus as well as several pathogenic events associated with coronavirus infection (Gordon et al., 2020, A SARS-CoV-2 protein interaction map reveals targets for drug repurposing [published online ahead of print, 2020 Apr 30]. Nature. 2020;10.1038/s41586-020-2286-9. doi:10.1038/s41586-020-2286-9). 108600 and 108110, unlike antiviral treatments developed in the past, do not target viral proteins, which can solve the problem of resistance to previously developed drugs.

In another aspect, provided herein are methods for treating a virus infection or a disease associated therewith, comprising administering a compound described herein to a subject in need thereof. In one embodiment, provided herein is a method for treating an RNA virus infection or a disease associated therewith comprising administering a compound described herein to a subject in need thereof. The RNA virus may be single stranded or double-stranded, positive or negative sense, and segmented or non-segmented. In some embodiments, the RNA virus is a single-stranded, positive sense RNA virus. In other embodiments, the RNA virus is a single-stranded, negative sense segmented or non-segmented virus. In a specific embodiment, the RNA virus is a coronavirus (e.g., SARS-CoV-1 or SARS-CoV-2), an influenza virus (e.g., an influena A virus or an influenza B virus), a hepatitis C virus, a vesicular stomatitis virus (VSV) or a Newcastle disease virus (NDV). In a particular embodiment, provided herein is a method for treating a SARS-CoV-2 infection or COVD-19, comprising administering a compound described herein to a subject in need thereof. A compound described herein may be provided in any acceptable format for veterinary or human administration. In a specific embodiment, the compound is a compound of Formula (I) or a salt thereof. See Section 4.1 for compounds of Formula (I). In another specific embodiment, the compound is 108600. In another specific embodiment, the compound is 108110.

In another embodiment, provided herein is a method for treating a virus infection or a disease associated therewith, comprising administering an effective amount of a compound described herein to a subject in need thereof. In another embodiment, provided herein is a method for treating an RNA virus infection or a disease associated therewith, comprising administering an effective amount of a compound described herein to a subject in need thereof. The RNA virus may be single stranded or double-stranded, positive or negative sense, and segmented or non-segmented. In some embodiments, the RNA virus is a single-stranded, positive sense RNA virus. In other embodiments, the RNA virus is a single-stranded, negative sense segmented or non-segmented virus. In a specific embodiment, the RNA virus is a coronavirus (e.g., SARS-CoV-1 or SARS-CoV-2), an influenza virus (e.g., an influena A virus or an influenza B virus), a heptatitis C virus, a vesicular stomatitis virus (VSV) or a Newcastle disease virus (NDV). In a particular embodiment, provided herein is a method for treating a SARS-CoV-2 infection or COVD-19, comprising administering an effective amount of a compound described herein to a subject in need thereof. A compound described herein may be provided in any acceptable format for veterinary or human administration. In a specific embodiment, the compound is a compound of Formula (I) or a salt thereof. See Section 4.1 for compounds of Formula (I). In another specific embodiment, the compound is 108600. In another specific embodiment, the compound is 108110.

In another embodiment, provided herein is a method for treating a virus infection or a disease associated therewith, comprising administering a pharmaceutical composition comprising an effective amount of a compound described herein to a subject in need thereof. In another embodiment, provided herein is a method for treating an RNA virus infection or a disease associated therewith, comprising administering a pharmaceutical composition comprising an effective amount of a compound described herein to a subject in need thereof. The RNA virus may be single stranded or double-stranded, positive or negative sense, and segmented or non-segmented. In some embodiments, the RNA virus is a single-stranded, positive sense RNA virus. In other embodiments, the RNA virus is a single-stranded, negative sense segmented or non-segmented virus. In a specific embodiment, the RNA virus is a coronavirus (e.g., SARS-CoV-1 or SARS-CoV-2), an influenza virus (e.g., an influena A virus or an influenza B virus), a hepatitis C virus, a vesicular stomatitis virus (VSV) or a Newcastle disease virus (NDV). In a particular embodiment, provided herein is a method for treating a SARS-CoV-2 infection or COVD-19, comprising administering a pharmaceutical composition comprising an effective amount of a compound described herein to a subject in need thereof. A compound described herein may be provided in any acceptable format for veterinary or human administration. In a specific embodiment, a compound described herein is a compound of Formula (I) or a salt thereof. See Section 4.1 for compounds of Formula (I). In another specific embodiment, the compound is 108600. In another specific embodiment, the compound is 108110.

In another aspect, provided herein are methods for preventing a disease associated with a virus infection, comprising administering a compound described herein to a subject in need thereof. In one embodiment, provided herein is a method for treating preventing a disease associated with an RNA virus infection, comprising administering a compound described herein to a subject in need thereof. The RNA virus may be single stranded or double-stranded, positive or negative sense, and segmented or non-segmented. In some embodiments, the RNA virus is a single-stranded, positive sense RNA virus. In other embodiments, the RNA virus is a single-stranded, negative sense segmented or non-segmented virus. In a specific embodiment, the RNA virus is a coronavirus (e.g., SARS-CoV-1 or SARS-CoV-2), an influenza virus (e.g., an influena A virus or an influenza B virus), a hepatitis C virus, a vesicular stomatitis virus (VSV) or a Newcastle disease virus (NDV). In a particular embodiment, provided herein is a method for preventing COVID-19, comprising administering a compound described herein to a subject in need thereof. A compound described herein may be provided in any acceptable format for veterinary or human administration. In a specific embodiment, the compound is a compound of Formula (I) or a salt thereof. See Section 4.1 for compounds of Formula (I). In another specific embodiment, the compound is 108600. In another specific embodiment, the compound is 108110.

In another embodiment, provided herein is a method for preventing a disease associated with a virus infection, comprising administering an effective amount of a compound described herein to a subject in need thereof. In another embodiment, provided herein is a method for preventing a disease associated with an RNA virus infection, comprising administering an effective amount of a compound described herein to a subject in need thereof. The RNA virus may be single stranded or double-stranded, positive or negative sense, and segmented or non-segmented. In some embodiments, the RNA virus is a single-stranded, positive sense RNA virus. In other embodiments, the RNA virus is a single-stranded, negative sense segmented or non-segmented virus. In a specific embodiment, the RNA virus is a coronavirus (e.g., SARS-CoV-1 or SARS-CoV-2), an influenza virus (e.g., an influena A virus or an influenza B virus), a hepatitis C virus, a vesicular stomatitis virus (VSV) or a Newcastle disease virus (NDV). In a particular embodiment, provided herein is a method for preventing COVD-19, comprising administering an effective amount of a compound described herein to a subject in need thereof. A compound described herein may be provided in any acceptable format for veterinary or human administration. In a specific embodiment, the compound is a compound of Formula (I) or a salt thereof. See Section 4.1 for compounds of Formula (I). In another specific embodiment, the compound is 108600. In another specific embodiment, the compound is 108110.

In another embodiment, provided herein is a method for preventing a disease associated with a virus infection, comprising administering a pharmaceutical composition comprising an effective amount of a compound described herein to a subject in need thereof. In another embodiment, provided herein is a method for preventing a disease associated with an RNA virus infection, comprising administering a pharmaceutical composition comprising an effective amount of a compound described herein to a subject in need thereof. The RNA virus may be single stranded or double-stranded, positive or negative sense, and segmented or non-segmented. In some embodiments, the RNA virus is a single-stranded, positive sense RNA virus. In other embodiments, the RNA virus is a single-stranded, negative sense segmented or non-segmented virus. In a specific embodiment, the RNA virus is a coronavirus (e.g., SARS-CoV-1 or SARS-CoV-2), an influenza virus (e.g., an influena A virus or an influenza B virus), a hepatitis C virus, a vesicular stomatitis virus (VSV) or a Newcastle disease virus (NDV). In a particular embodiment, provided herein is a method for preventing COVD-19, comprising administering a pharmaceutical composition comprising an effective amount of a compound described herein to a subject in need thereof. A compound described herein may be provided in any acceptable format for veterinary or human administration. In a specific embodiment, the compound is a compound of Formula (I) or a salt thereof. See Section 4.1 for compounds of Formula (I). In another specific embodiment, the compound is 108600. In another specific embodiment, the compound is 108110.

### DESCRIPTION OF THE FIGURES

FIG. 1. Structure of ON108600.

FIG. 2. Structure of ON108110.

FIGS. 3A-3D. Effect of Remdesevir (FIG. 3A), Hydroxychloroquine (FIG. 3B), 108110 (FIG. 3C), and 108600 (FIG. 3D) on COVID-19 replication and cell viability.

FIGS. 4A-4D. Inhibition of COVID-19 infection in Vero E6 cells at high cell density in presence of remdesivir.

FIGS. 5A - 5D. Effect of cell cycle inhibitors 108110 and 108600 on COVID-19 infection at high cell density.

FIGS. 6A - 6B. Inhibition of COVID-19 infection at low cell density in presence of remdesivir.

FIGS. 7A - 7B. Effect of cell cycle inhibitor 108110 on COVID-19 infection at low cell density.

### DETAILED DESCRIPTION

### 4.1 Compounds

Provided herein are compounds of Formula (I), or a salt thereof:

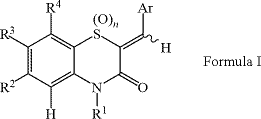

wherein
n is 0, 1, or 2,
R¹ is selected from the group consisting of -H, -(C₁-C₆)alkyl, -(C₂-C₆)alkenyl, -(C₂-C₆)alkynyl, optionally substituted aryl, optionally substituted heteroaryl, optionally substituted aryl-(C₁-C₆)alkyl, optionally substituted heteroaryl-(C₁-C₆)alkyl, —C(═O)(C₁-C₆)alkyl, -C(=O)(C₂-C₆)alkenyl, —C(═O)— optionally substituted aryl, —C(═O)(CH₂)ₘ-optionally substituted aryl, and —C(═O)(CH₂)ₚ—optionally substituted heteroaryl;
R², R³, and R⁴ are independently selected from the group consisting of -H, halogen, —CN, -NR¹⁰R¹¹, —OH, -OR¹³, -(C₁-C₆)alkoxy, —NO₂, -(C₁-C₆₎alkyl, -(C₁-C₆)perfluoroalkyl, -(C₁-C₆)perfluoroalkoxy, -C(=O)R¹⁵, -C(=O)OR¹⁵, -OC(=O)R¹², -OC(=O)OR¹² -C(=O)NR¹⁷R¹⁸, —SH,-S(C₁-C₆)alkyl, -SR¹³, -S(=O)R¹³, -S(=O)₂R¹³, -OS(=O)₂R¹³, -S(=O)_{q}R¹⁵, -OS(=O)_{q}R¹⁵, -S(=O)₂NR¹⁷R¹⁸, -S(=O)NR¹⁷R¹⁸, optionally substituted aryl, optionally substituted aryl-(C₁-C₆)alkyl, optionally substituted heteroaryl, optionally substituted heteroaryl-(C₁-C₆)alkyl, optionally substituted (C₂-C₉)heterocyclyl, optionally substituted (C₂-C₉)heterocyclyl-(C₁-C₆)alkyl, -NH(CH₂)ₘC(=O)OR -C(=NR¹⁴)NR¹⁴₂, -C(=N-OR¹⁴)NR¹⁴₂,-P(=O)(OR¹⁴)₂. and -OP(=O)(OR¹⁴)₂;
Ar is optionally substituted heteroaryl, optionally substituted (C₁₀-C₁₄)aryl, or

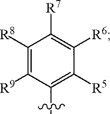

R⁵, R⁶, R⁷, R⁸, and R⁹ are independently selected from the group consisting of -H, —OH, -OR¹³, —NO₂, halogen, —CN, -NR¹⁰R¹¹, -(CH₂)ₘNR¹⁰R¹¹, -O(CH₂)ₘNR¹⁰R¹¹, -(C₁-C₆)alkyl, -(CH₂)mO(C₁-C₆)alkyl, -(C₁-C₆)alkoxy, -(C₁-C₆)perfluoroalkyl, (C₁-C₆)perfluoroalkoxy, —SH, -S(C₁-C₆)alkyl, -SR¹³, -S(=O)R¹⁵, -S(=O)₂R¹⁵, -C(=O)R¹⁵, -C(=O)OR¹⁵, -C(=O)NR¹⁷R¹⁸, -OC(=O)R¹⁶, -OC(=O)OR¹², -OC(=O)NR¹⁷R¹⁸, heterocyclyl, optionally substituted heteroaryl, -NH(CH₂)ₘC(=O)OR^{14,} -OS(=O)₂R¹⁶, -C(=NR¹⁴)NR¹⁴₂, -C(=N-OR¹⁴)NR¹⁴₂, -P(=O)(OR¹⁴)₂, and -OP(=O)(ORI¹⁴)_{2;}
each R¹⁰ and R¹¹ is independently selected from the group consisting of -H, -(C₁-C₆)alkyl, -(C₁-C₆)alkoxy, -C(=O)R¹², -C(C=O)NR¹⁷R¹⁸, -C(=O)OR¹², -C(=NR¹⁴)NR¹⁷R¹⁸, R¹³, optionally substituted aryl, optionally substituted heteroaryl, and -C(=NR¹⁴)R¹⁵, or R¹⁰ and R¹¹, together with the nitrogen to which they are bound, form an optionally substituted (C₂-C₅)heterocycle,
each R¹² is independently selected from the group consisting of -(C₁-C₆,)alkyl, and optionally substituted aryl;
each R¹³ is independently selected from the group consisting of optionally substituted aryl and -(CH₂)ₘR¹⁶;
each R¹⁴ is independently selected from the group consisting of -H and -(C₁-C₆)alkyl; or two occurrences of R¹⁴ bound to the same nitrogen form a (C₂-C₆)heterocycle, together with the nitrogen atom to which they are bound,
each R¹⁵ is independently selected from the group consisting of -H, -(C₁-C₆)alkyl, optionally substituted aryl, and NR¹⁴₂;
each R¹⁶ is independently selected from the group consisting of -(C₁-C₆)alkyl, -NR¹⁴₂, and Ar¹;
each R¹⁷ and R¹⁸ is independently selected from the group consisting of -H, -(C₁-C₆)alkyl, -(C₁-C₆)alkoxy, R¹³, optionally substituted aryl, and optionally substituted heteroaryl; or R¹⁷ and R¹⁸, together with the nitrogen to which they are bound, form an optionally substituted (C₂-C₃)heterocycle;
m is independently at each occurrence 1, 2, 3, 4, or 5;
p is independently at each occurrence 0, 1, 2, or 3,
q is independently at each occurrence 0, 1, or 2;
each optionally substituted aryl, optionally substituted (C₁₀)-C₁₄)aryl, optionally substituted heteroaryl, optionally substituted aryl-(C₁-C₆,)alkyl, optionally substituted heteroaryl-(C₁-C₆)alkyl, optionally substituted (C₂-C₉)heterocyclyl, optionally substituted (C₂-C₉)heterocyclyl-(C₁-C₆)alkyl, and optionally substituted (C₂-C₅)heterocycle is optionally substituted with one or more substituents independently selected from the group consisting of halogen, —CN, -NR¹⁴₂, -(CH₂)mNR¹⁴₂, -O(CH₂)mNR¹⁴₂, -NR¹⁴C(=O)(C₁-C₆)alkyl, -NR¹⁴C(=O)O(C₁-C₆)alkyl, -NR¹⁴C(=O)NR¹⁴₂, -NR¹⁴C(=NR₁₄)NR¹⁴₂, -NH(CH₂)ₘC(=O)OR¹⁴, —OH, —NO₂, -(C₁-C₆)alkyl, -(CH₂)ₘO(C₁-C₆)alkyl, -(C₁-C₆)alkoxy, (C₂-C₆)alkenyl, -(C₂-C₆)alkynyl, -SR¹⁴, -S(=O)R¹⁵, -S(=O)₂R¹⁵, -NR¹⁴S(=O)₂R¹⁵, -(C₁-C₆)perfluoroalkyl, -(C₁-C₆)perfluoroalkoxy, -C(=O)R¹⁴, -C(=O)OR¹⁴, -C(=O)NR¹⁴₂, -OC(=O)R¹⁴, -OC(=O)NR¹⁴₂, -OC(=O)O(C₁-C₆)alkyl, -P(=O)(OR¹⁴)₂, -OP(=O)(OR¹⁴)₂, heterocyclyl, and heteroaryl;
Ar¹ is a radical according to Formula II

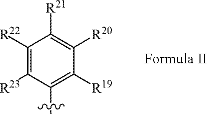

wherein R¹⁹, R²⁰, R²¹, R²², and R²³ are independently selected from the group consisting of —H, —OH, —NO₂, halogen, —CN, -NR¹⁰R¹¹, -(CH₂)ₘNR¹⁰R¹¹, -O(CH₂)mNR¹⁰R¹¹, -(C₁-C₆)alkyl, -(CH₂)ₘO(C₁-C₆₎alkyl, -(C₁-C₆)alkoxy, -(C₁-C₆)perfluoroalkyl, -(C₁-C₆)perfluoroalkoxy, —SH, -SR¹², -(=O)R¹⁵, -S(=O)₂R¹⁵, -C(=O)R¹⁵, -C(=O)OR¹⁵, -C(=O)NR¹⁷R¹⁸, -OC(=O)R¹⁶, -OC(=O)OR¹², -OC(=O)NR¹⁷R¹⁸, heterocyclyl, optionally substituted heteroaryl, -NH(CH₂)ₘC(=O)OR¹⁴, -OS(=O)₂R¹⁶, -C(=NR¹⁴)NR¹⁴₂, -C(N-OR¹⁴)NR¹⁴₂, -P(=O)(OR¹⁴)_{2,} and -OP(=O)(OR¹⁴)₂;
provided that:
   i) at least one of R², R³, or R⁴ is other than hydrogen;
   ii) when none of R², R³, and R⁴ are -OR¹³, -NHR¹³, -SR¹³, -S(=O)R¹³, or -S(=O)₂R¹³, and Ar is 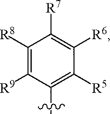
   then at least one of R⁶ and R⁸ is —NO₂ and at least R⁷ is other than hydrogen or halogen; and
   iii) when Ar is optionally substituted heteroaryl and none of R², R³; or R⁴ are -OR¹³⁻, -NHR¹³, -SR¹³, -S(=O)R¹³, or -S(=O)₂R¹³, then R¹ is other than hydrogen.

In preferred embodiments of a compound of Formula I, n is 0. In other embodiments, however, n is 1 or 2.

The wavy bond in the structure of Formula I indicates either (E), (Z), or a mixture of configurations of the double bond to the carbon atom to which Ar and

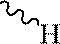

are attached.

In certain embodiments of the invention, the double bond in the compounds of Formula I can be in the E configuration In preferred embodiments, the double bond is in the Z configuration:

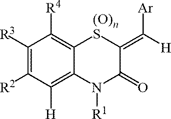

In certain instances, the double bond in the compound of Formula I is in the Z configuration, n is 0, and Ar is optionally substituted heteroaryl or

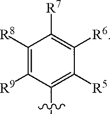

In certain embodiments, the optionally substituted heteroaryl group can be thiophene-2-yl (thiene-2-yl), thiophene-3-yl (thiene-3-yl), indol-2-yl, indol-3-yl, indol-4-yl, indol-5-yl, indol-6-yl, indol-7-yl, pyrrol-2-yl, pyrrol-3-yl, pyrimidin-4-yl, pyrimidin-5-yl, or pyrimidin-6-yl, any of which can be optionally substituted In certain embodiments, the thiophene-2-yl (thiene-2-yl), thiophene-3-yl (thiene-3-yl), indol-2-yl, indol-3-yl, indol-4-yl, indol-5-yl, indol-6-yl, indol-7-yl, pyrrol-2-yl, pyrrol-3-yl, pyrimidin-4-yl, pyrimidin-5-yl, and pyrimidin-6-yl radicals can be optionally substituted with a halogen, an alkyl group, such as methyl or ethyl, or an acyl group such as an acetyl group. In particular embodiments, the acetyl group can be present on the nitrogen of any of the pyrrolyl or indolyl radicals. In other instances, the pyrimidinyl radicals can be substituted with a thioether, such as —SCH₃, or a morpholino group at any of the substitutable position, and in particular embodiments, at the 2 position of the pyriminidyl radical.

In certain embodiments of the invention, any of R², R³, or R⁴ can be -S(=O)₂R¹³ wherein R¹³ is -(CH₂)ₘR¹⁶, m is 1, and R¹⁶ is

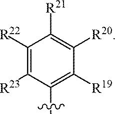

In particular embodiments, R² is -S(=O)₂R¹³ wherein R¹³ is -CH₂)ₘR¹⁶, m is 1, and R¹⁶ is

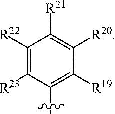

In certain embodiments, at least two of R¹⁹, R²⁰, R²¹, R²², and R²³ are halogen atoms and can be the same or different halogen atoms. In particular embodiments, R¹⁹ and R²³ are both halogen atoms (either the same or different), while R²⁰, R²¹, and R²² are other than halogen. In a further embodiment, R¹⁹ and R²³ are the same halogen atom, while R²⁰. R²¹, and R²² are other than halogen. In certain embodiments, R19 and R²² are both chlorine atoms and R^{20,} R²¹, and R²² are each hydrogen.

Particular examples of compounds according to the invention, and salts thereof, are set forth in Table 1:

TABLE 1

| | |
|---|---|
| Cpd. # Name | Structure |
| 1 (Z)-4-((6-chloro-3-oxo-3,4-dihydro-2H-benzo[b][1,4]thiazin-2-ylidene)methyl)-2-nitrophenyl acetate | 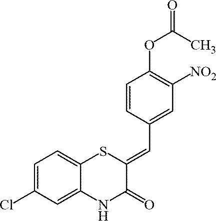 [SMILES: CC(=O)Oc1ccc(/C=C2\Sc3ccc(Cl)cc3NC2=O)cc1[N+](=O)[O-]] |
| 2 (Z)-4-((7-methoxy-3-oxo-3,4-dihydro-2H-benzo[b][1,4]thiazin-2-ylidene)methyl)-2-nitrophenyl acetate | 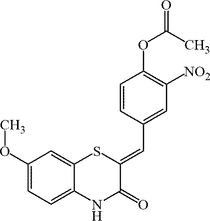 [SMILES: COc1ccc2c(c1)S/C(=C\c1ccc(OC(C)=O)c([N+](=O)[O-])c1)C(=O)N2] |

TABLE 1-continued

| | |
|---|---|
| Name | Structure |
| 3 (Z)-4-((7--3,4- dihydro-2H-ylidem)methyl)-2- | 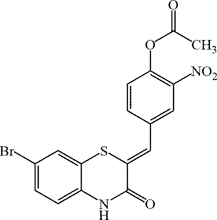 [SMILES: CC(=O)Oc1ccc(/C=C2\Sc3cc(Br)ccc3NC2=O)cc1[N+](=O)[O-]] |
| 4 (Z)-4-((7-methyl-3-oxo-3,4 dihydro-2H-benzo[b][1,4]thiazin-2-yildene-metyl)-2-nitrophenyl acetate | 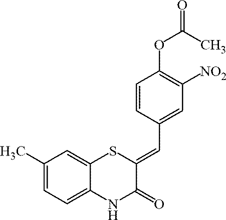 [SMILES: CC(=O)Oc1ccc(/C=C2\Sc3cc(C)ccc3NC2=O)cc1[N+](=O)[O-]] |
| 5 (Z)-2-(4-acetoxy-1-nitrophenylidene)-3-oxo-3,4-dihydro-2H benxo[b][1,4]thiazine-7-carboxylic acid | 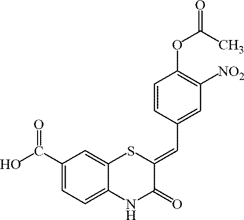 [SMILES: CC(=O)Oc1ccc(/C=C2\Sc3cc(C(=O)O)ccc3NC2=O)cc1[N+](=O)[O-]] |
| 6 (Z)-4-((7-fluro-3-oxo 3,4 dihdydro-2H-benzo[b][1,4]thiazin-2-ylideae)methyl)-nitrophenyl acetate | 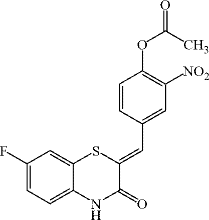 [SMILES: CC(=O)Oc1ccc(/C=C2\Sc3cc(F)ccc3NC2=O)cc1[N+](=O)[O-]] |

TABLE 1-continued

| | |
|---|---|
| Name | Structure |
| (Z)-4-((6-fluoxo-3-oxo-3,4 dihydro-2H-benzo[b][1,4]thiazine-2-ylidenmethyl)-2-ntrophenyl acetate | 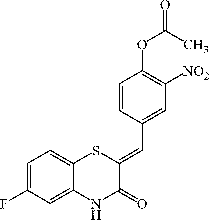 [SMILES: CC(=O)Oc1ccc(/C=C2\Sc3ccc(F)cc3NC2=O)cc1[N+](=O)[O-]] |
| 8 (Z)-4-((6-((2,6-dihydr-3oxo-3,4-dihydro-2H-benzo[b][1,4]thiazin-2-ylideaemthyl)-2-nitrophenyl acetate | 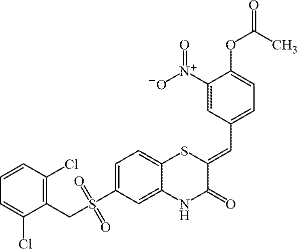 [SMILES: CC(=O)Oc1ccc(/C=C2\Sc3ccc(S(=O)(=O)Cc4c(Cl)cccc4Cl)cc3NC2=O)cc1[N+](=O)[O-]] |
| 9 (Z)-4-((6-((3,6--dihydro-2H-benzo[b][1,4]thiazin-3-yliden)methylphenyl acetate | 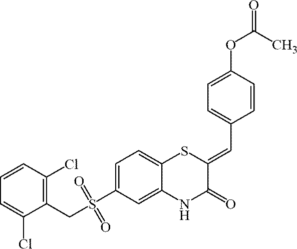 [SMILES: CC(=O)Oc1ccc(/C=C2\Sc3ccc(S(=O)(=O)Cc4c(Cl)cccc4Cl)cc3NC2=O)cc1] |
| 10. (Z) dihydroxobenzyl)sulfanyl)-2H-benzo[b][3,4]thiazin-3(4H)-oxo | 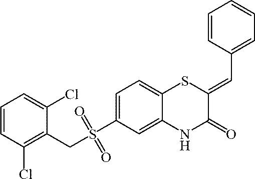 [SMILES: O=C1Nc2cc(S(=O)(=O)Cc3c(Cl)cccc3Cl)ccc2S/C1=C\c1ccccc1] |

TABLE 1-continued

| | |
|---|---|
| Cpd. Name | Structure |
| 11 2-(4-3 [b][1,4] | 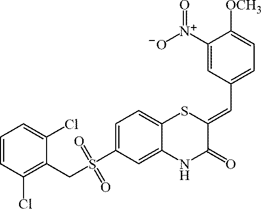 [SMILES: COc1ccc(/C=C2\Sc3ccc(S(=O)(=O)Cc4c(Cl)cccc4Cl)cc3NC2=O)cc1[N+](=O)[O-]] |
| 12 (Z)-2-(3-amino-4-methoxybenzylidone)-6-((2,6- | 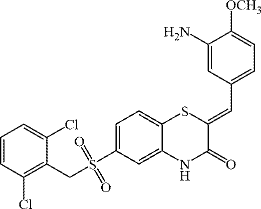 [SMILES: COc1ccc(/C=C2\Sc3ccc(S(=O)(=O)Cc4c(Cl)cccc4Cl)cc3NC2=O)cc1N] |
| 13 (Z)-6-((2,6-dichlorobenzyl yl )-2H- | 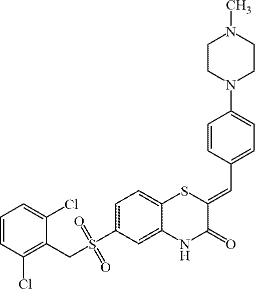 [SMILES: CN1CCN(c2ccc(/C=C3\Sc4ccc(S(=O)(=O)Cc5c(Cl)cccc5Cl)cc4NC3=O)cc2)CC1] |
| 14 (Z)-6-((2,6-dichlorobenzyl) | 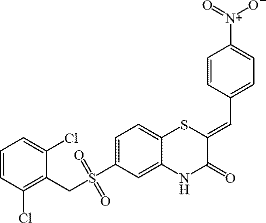 [SMILES: O=C1Nc2cc(S(=O)(=O)Cc3c(Cl)cccc3Cl)ccc2S/C1=C\c1ccc([N+](=O)[O-])cc1] |

TABLE 1-continued

| | |
|---|---|
| Cpd. Name | Structure |
| 15 (Z)-3-(4-aminobenzylylene)-6- ((2,6- )-2H-benzo[b][1,4] )(4H)-one | 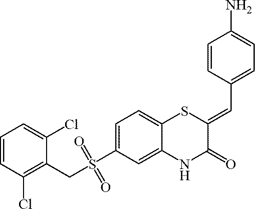 [SMILES: Nc1ccc(/C=C2\Sc3ccc(S(=O)(=O)Cc4c(Cl)cccc4Cl)cc3NC2=O)cc1] |
| 16 (Z)-6-((2,6-dichlorobenzyl)sulfoxyl)-2-(4-benzo[b][1,4] )(4H)- | 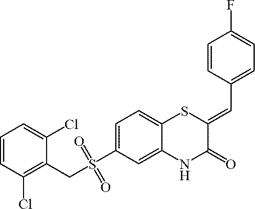 [SMILES: O=C1Nc2cc(S(=O)(=O)Cc3c(Cl)cccc3Cl)ccc2S/C1=C\c1ccc(F)cc1] |
| 17 (Z)-2-(4-chlorobenzylidone)-6- ((2,6-dichlorobenzyl)sulfoxyl)- 2H-benzo[b][1,4] one | 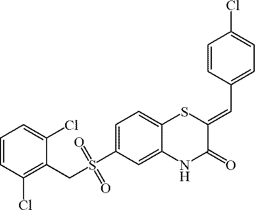 [SMILES: O=C1Nc2cc(S(=O)(=O)Cc3c(Cl)cccc3Cl)ccc2S/C1=C\c1ccc(Cl)cc1] |
| 18 (Z)-2-(42- 2H- 3(4H)-one | 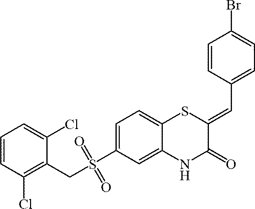 [SMILES: O=C1Nc2cc(S(=O)(=O)Cc3c(Cl)cccc3Cl)ccc2S/C1=C\c1ccc(Br)cc1] |

TABLE 1-continued

| | |
|---|---|
| Cpd. Name | Structure |
| 19 (Z)-6-((2,6-dichlorobenzyl)sulfoxyl)-2-(4-methoxybenzylidene)-2H-benzo[b][1,4] | 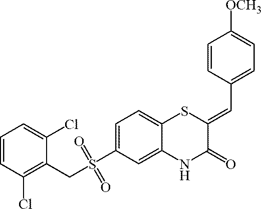 [SMILES: COc1ccc(/C=C2\Sc3ccc(S(=O)(=O)Cc4c(Cl)cccc4Cl)cc3NC2=O)cc1] |
| 20 (Z)-6-((2,6-dichlorobenzyl)sulfoxyl)- 2-(4-methylbenzylidene)-2H-benzo[b][1,4] | 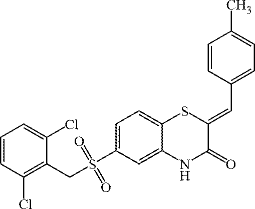 [SMILES: Cc1ccc(/C=C2\Sc3ccc(S(=O)(=O)Cc4c(Cl)cccc4Cl)cc3NC2=O)cc1] |
| 21 (Z)-6-((2,6-dichlorobenzyl)sulfoxyl)-2-(2,4,6-trimethoxybenzylilene)-2H-benzo[b][1,4]thiazin)(4H)-one | 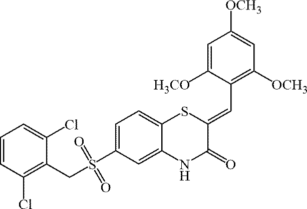 [SMILES: COc1cc(OC)c(/C=C2\Sc3ccc(S(=O)(=O)Cc4c(Cl)cccc4Cl)cc3NC2=O)c(OC)c1] |
| 22 (Z)-6-((2,6-dichlorobenzyl)sulfoxyl)-2-(2,4-dichlorobenzylidene)-2H-benzo[b][1,4]thiazin-3(4H)-one | 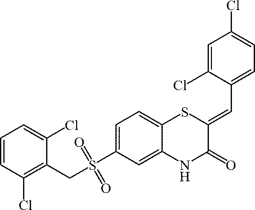 [SMILES: O=C1Nc2cc(S(=O)(=O)Cc3c(Cl)cccc3Cl)ccc2S/C1=C\c1ccc(Cl)cc1Cl] |

TABLE 1-continued

| | |
|---|---|
| Cpd. Name | Structure |
| 24 (Z)-methyl-2-((4-((6-((2,6-dichlorobenzyl)sulfoxyl)-3-oxo-3,4-dyhydro-2H-benzo[b][1,4]thiazin-2-ylidene)methyl)phenyl)amino) acetate | 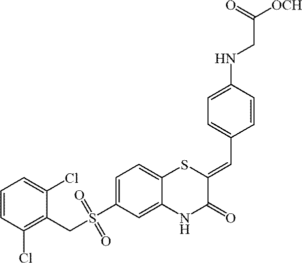 |
| 25 (Z)-2-((4-((6-((2,6-dichlorobenzyl)sulfoxyl)-3-oxo-3,4-dihydro-2H-benzo[b][1,4]thiazin-2-ylidene)methyl)phenyl)amino) acetic acid | 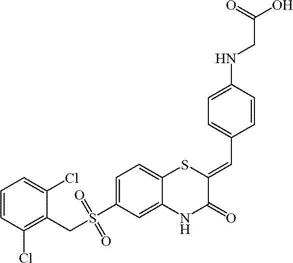 [SMILES: O=C(O)CNc1ccc(/C=C2\Sc3ccc(S(=O)(=O)Cc4c(Cl)cccc4Cl)cc3NC2=O)cc1] |
| 26 yl)methylene)-6-((2,6-dichlorobenzyl)sulfoxyl)-2H-benzo[b][1,4 ]thiazin-3(4H)-one | 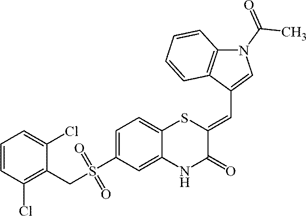 [SMILES: CC(=O)n1cc(/C=C2\Sc3ccc(S(=O)(=O)Cc4c(Cl)cccc4Cl)cc3NC2=O)c2ccccc21] |
| 27 (Z)-2-((1H-pyrrol-3-yl)methylene)-6-((2,6-dichlorobenzyl)sulfoxyl)-2H-benzo[b][1,4]thiazin-3(4H)-one | 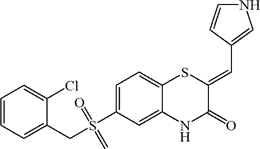 [SMILES: C=S(=O)(Cc1ccccc1Cl)c1ccc2c(c1)NC(=O)/C(=C/c1cc[nH]c1)S2] |

TABLE 1-continued

| | |
|---|---|
| Cpd. Name | Structure |
| 28 (Z)-6-((2,6-dichlorobenzyl)sulfoxyl)-2-(4-hydroxy-2,6-dimethoxybenzylidene)-2H-benzo[b][1,4]thiazin-3(4H)-one | 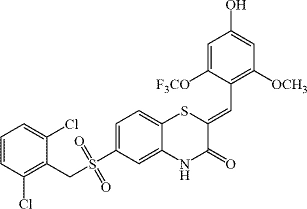 [SMILES: COc1cc(O)cc(OC(F)(F)F)c1/C=C1\Sc2ccc(S(=O)(=O)Cc3c(Cl)cccc3Cl)cc2NC1=O] |
| 29 (Z)-2-(4-chloro-3- 2,6-dichlorobenzyl)sulfoxyl)-2H-benzo[b][1,4]thiazin-3(4H)-one | 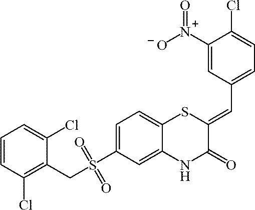 [SMILES: O=C1Nc2cc(S(=O)(=O)Cc3c(Cl)cccc3Cl)ccc2S/C1=C\c1ccc(Cl)c([N+](=O)[O-])c1] |
| 30 (Z)-6-((2,6-dichlorobenzyl)sulfoxyl)-2-(2,4-d benzo[b][1,4]thiazin-3(4H)-one | 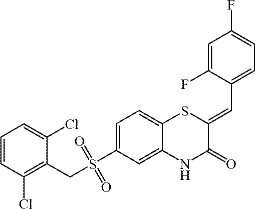 [SMILES: O=C1Nc2cc(S(=O)(=O)Cc3c(Cl)cccc3Cl)ccc2S/C1=C\c1ccc(F)cc1F] |
| 31 (Z)-6-((2,6-dichlorobenzyl)sulfoxyl)-2-(2,4,6-trifluorobenzylidene)-2H benzo[b][1,4]thiazin-3(4H)-one | 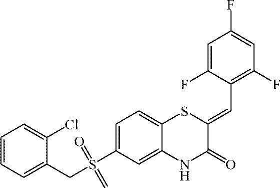 [SMILES: C=S(=O)(Cc1ccccc1Cl)c1ccc2c(c1)NC(=O)/C(=C/c1c(F)cc(F)cc1F)S2] |

TABLE 1-continued

| | |
|---|---|
| Cpd. Name | Structure |
| 32 (Z)-methyl-4-((6-((2,6-dichlorobenzyl)sulfoxyl)-3-oxo 3,4-dihydro-2H-benzo[b][1,4]thiazin-2-ylidene)methyl)benzoste | 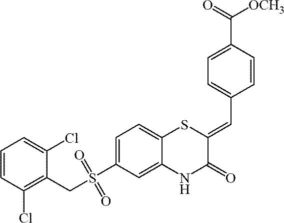 [SMILES: COC(=O)c1ccc(/C=C2\Sc3ccc(S(=O)(=O)Cc4c(Cl)cccc4Cl)cc3NC2=O)cc1] |
| 33 (Z)-6-((2,6-dichlorobenzyl)sulfoxyl)-2-(thiophen-2-ylmethylene)-2H- benzo[b][1,4]thiazin-3(4H)-one | 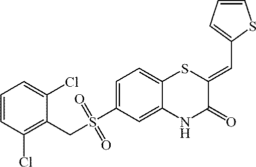 [SMILES: O=C1Nc2cc(S(=O)(=O)Cc3c(Cl)cccc3Cl)ccc2S/C1=C\c1cccs1] |
| 34 (Z)-2-((1H-indol-3- yl)methylene)-6-((2,6-dichlorobenzyl)sulfoxyl)-2H-benzo[b][1,4]thiazin-3(4H)-one | 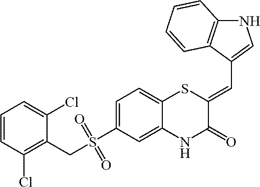 [SMILES: O=C1Nc2cc(S(=O)(=O)Cc3c(Cl)cccc3Cl)ccc2S/C1=C\c1c[nH]c2ccccc12] |
| 35 (Z)-methyl-4-((6-((2,6-dichlorobenzyl)sulfoxyl)-3-oxo-3,4-dihydro-2H-benzo[b][1,4]thiazin-2-ylidene )methyl)-3-nitrobenzoate | 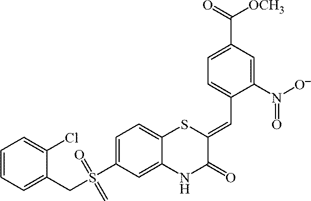 |

TABLE 1-continued

| | |
|---|---|
| Cpd. Name | Structure |
| 36 (Z)-6((2,6-dichlorobenzyl)sulfoxyl)-2-((2-(methylthio)pyrimidin-4-yl)methylene)-2H-benzo[b][1,4]thiazin-3(4H)-one | 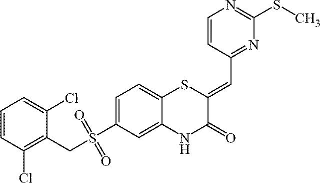 [SMILES: CSc1nccc(/C=C2\Sc3ccc(S(=O)(=O)Cc4c(Cl)cccc4Cl)cc3NC2=O)n1] |
| 37 (Z)-6((2,6-dichlorobenzyl)sulfoxyl-2-((2-yl)methylene)-2H-benzo[b][1,4]thiazin-3(4H)-one | 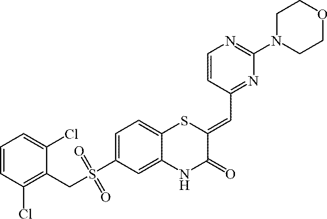 [SMILES: O=C1Nc2cc(S(=O)(=O)Cc3c(Cl)cccc3Cl)ccc2S/C1=C\c1ccnc(N2CCOCC2)n1] |
| 38 (Z)-4-((6-((2,6-dichlorobenzyl)sulfoxyl)-3-oxo-3,4-dihydro-2H-benzo[b][1,4]thiazin -2-ylidene)methyl)-2-nitrophenyl 4-methylbenzenesulfoxate | 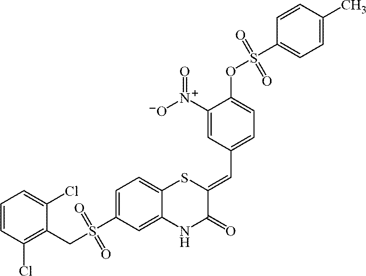 [SMILES: Cc1ccc(S(=O)(=O)Oc2ccc(/C=C3\Sc4ccc(S(=O)(=O)Cc5c(Cl)cccc5Cl)cc4NC3=O)cc2[N+](=O)[O-])cc1] |
| 39 (Z)-2(4-(2H- yl)benzylidene)-6-((2,6-dichlorobenzyl)sulfoxyl)-2H-benzo[b][1,4]thiazin-3(4H)-one | 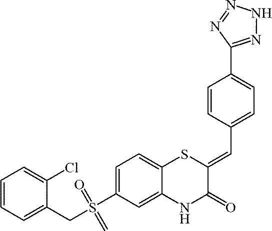 [SMILES: C=S(=O)(Cc1ccccc1Cl)c1ccc2c(c1)NC(=O)/C(=C/c1ccc(-c3nn[nH]n3)cc1)S2] |

TABLE 1-continued

| | |
|---|---|
| Cpd. Name | Structure |
| 40 (Z)-4-((4-acetyl-2-methoxy-3-oxo-3,4-dihydro-2H-benzo[b][1,4]thiazin-2-ylidene)methyl)-2-nitrophenyl acetate | 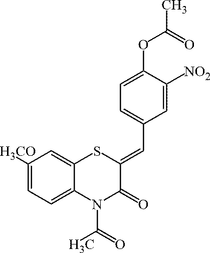 [SMILES: COc1ccc2c(c1)S/C(=C\c1ccc(OC(C)=O)c([N+](=O)[O-])c1)C(=O)N2C(C)=O] |
| 41 (Z)-2-(4-(benzyloxy)-3-nitrobenzylidene)-6-((2,6-dichlorobenzyl)sulfoxyl)-2H-benzo[b][1,4]thiazin-3(4H)-one | 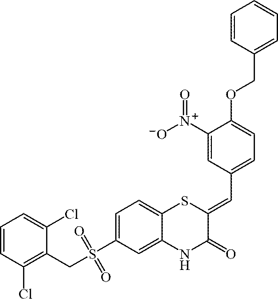 [SMILES: O=C1Nc2cc(S(=O)(=O)Cc3c(Cl)cccc3Cl)ccc2S/C1=C\c1ccc(OCc2ccccc2)c([N+](=O)[O-])c1] |
| 42 (Z)-6-((2,6-dichlorobenzyl)sulfoxyl)-2-(4-dichlorobenzyl)sulfoxyl)-2-(4-hydroxy-3nitrobenzylidene)- 2H1benzo[b][1,4]thiazin-3(4H)- one | 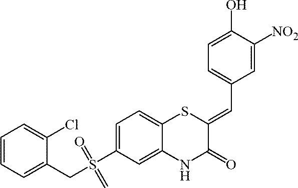 [SMILES: C=S(=O)(Cc1ccccc1Cl)c1ccc2c(c1)NC(=O)/C(=C/c1ccc(O)c([N+](=O)[O-])c1)S2] |

TABLE 1-continued

| | |
|---|---|
| Cpd. Name | Structure |
| 43 (Z)-2-(4-(benzyloxy)-3-nitrobenzylidene)-7-fluoro-2H-benzo[b][1,4]thiazin-3(4H)-one | 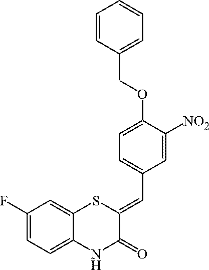 [SMILES: O=C1Nc2ccc(F)cc2S/C1=C\c1ccc(OCc2ccccc2)c([N+](=O)[O-])c1] |
| 44 (Z)-7-fluoro-2-(4-hydroxy-3-nitrobenzylidene)-2H-benzo[b][1,4]thiazin-3(4H)-one | 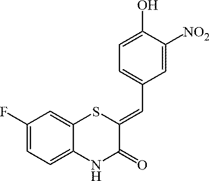 [SMILES: O=C1Nc2ccc(F)cc2S/C1=C\c1ccc(O)c([N+](=O)[O-])c1] |

Other exemplary compounds include, but are not limited to the following, and salts thereof. (Z)-4-((6-((2,6-dibromobenzyl)sulfonyl)-3-oxo-3,4-dihydro-2H-benzo[b][1,4]thiazin-2-ylidene)methyl)-2-nitrophenyl acetate; (Z)-4-((6-((2,6-dibromobenzyl)sulfonyl)-3-oxo-3,4-dihydro-2H-benzo[b][1,4]thiazin-2-ylidene)methyl)phenyl acetate; (Z)-2-benzylidene-6-((2,6-dibromobenzyl)sulfonyl)-2H-benzo[b][1,4]thiazin-3(4H)-one; (Z)-6-((2,6-dibromobenzyl)sulfonyl)-2-(4-methoxy-3-nitrobenzylidene)-2H-benzo[b][1,4]thiazin-3(4H)-one; (Z)-2-(3-amino-4-methoxybenzylidene)-6-((2,6-dibromobenzyl)sulfonyl)-2H-benzo[b][1,4]thiazin-3(4H)-one; (Z)-6-((2,6-dibromobenzyl)sulfonyl)-2-(4-(4-methylpiperazin-1-yl)benzylidene)-2H-benzo[b][1,4]thiazin-3(4H)-one; (Z)-6-((2,6-dibromobenzyl)sulfonyl)-2-(4-nitrobenzylidene)-2H-benzo[b][1,4]thiazin-3(4H)-one; (Z)-2-(4-aminobenzylidene)-6-((2,6-dibromobenzyl)sulfonyl)-2H-benzo[b][1,4]thiazin-3(4H)-one; (Z)-6-((2,6-dibromobenzyl)sulfonyl)-2-(4-fluorobenzylidene)-2H-benzo[b][1,4]thiazin-3(4H)-one; (Z)-2-(4-chlorobenzylidene)-6-((2,6-dibromobenzyl)sulfonyl)-2H-benzo[b][1,4]thiazin-3(4H)-one; (Z)-2-(4-bromobenzylidene)-6-((2,6-dibromobenzyl)sulfonyl)-2H-benzo[b][1,4]thiazin-3(4H)-one; (Z)-6-((2,6-dibromobenzyl)sulfonyl)-2-(4-methoxybenzylidene)-2H-benzo[b][1,4]thiazin-3(4H)-one, (Z)-6-((2,6-dibromobenzyl)sulfonyl)-2-(4-methylbenzylidene)-2H-benzo[b][1,4]thiazin-3(4H)-one; (Z)-6-((2,6-dibromobenzyl)sulfonyl)-2-(2,4,6-trimethoxybenzylidene)-2H-benzo[b][1,4]thiazin-3(4H)-one; (Z)-6-((2,6-dibromobenzyl)sulfonyl)-2-(2,4-dibromobenzylidene)-2H-benzo[b][1,4]thiazin-3(4H)-one; (Z)-6-((2,6-dibromobenzyl)sulfonyl)-2-(2,4,6-trifluorobenzylidene)-2H-benzo[b][1,4]thiazin-3(4H)-one; (Z)-methyl 2-((4-((6-((2,6-dibromobenzyl)sulfonyl)-3-oxo-3,4-dihydro-2H-benzo[b][1,4]thiazin-2-ylidene)methyl)phenyl)amino)acetate; (Z)-2-((4-((6-((2,6-dibromobenzyl)sulfonyl)-3-oxo-3,4-dihydro-2H-benzo[b][1,4]thiazin-2-ylidene)methyl)phenyl)amino)acetic acid; (Z)-6-((2,6-dibromobenzyl)sulfonyl)-2-(4-hydroxy-2,6-dimethoxybenzylidene)-2H-benzo[b][1,4]thiazin-3(4H)-one, (Z)-2-(4-chloro-3-nitrobenzylidene)-6-((2,6-dibromobenzyl)sulfonyl)-2H-benzo[b][1,4]thiazin-3(4H )-one; (Z)-6-((2,6-dibromobenzyl)sulfonyl)-2-(2,4-difluorobenzylidene)-2H-benzo[b][1,4]thiazin-3(4H)-one; (Z)-6-((2,6-dibromobenzyl)sulfonyl)-2-(2,4,6-trifluorobenzylidene)-2H-benzo[b][1,4]thiazin-3(4H)-one; (Z)-methyl 4-((6-((2,6-dibromobenzyl)sulfonyl)-3-oxo-3,4-dihydro-2H-benzo[b][1,4]thiazin-2-ylidene)methyl)benzoate; (Z)-methyl 4-((6-((2,6-dibromobenzyl)sulfonyl)-3-oxo-3,4-dihydro-2H-benzo[b][1,4]thiazin-2-ylidene)methyl)-3-nitrobenzoate; (Z)-4-((6-((2,6-dibromobenzyl)sulfonyl)-3-oxo-3,4-dihydro-2H-benzo[b][1,4]thiazin-2-ylidene)methyl)-2-nitrophenyl 4-methylbenzenesulfonate; (Z)-2-(4-(2H-tetrazol-5-yl)benzylidene)-6-((2,6-dibromobenzyl)sulfonyl)-2H-benzo[b][1,4]thiazin-3(4H)-one; (Z)-2-(4-(benzyloxy)-3-nitrobenzylidene)-6-((2,6-dibromobenzyl)sulfonyl)-2H-benzo[b][1,4]thiazin-3(4H)-one; (Z)-6-((2,6-dibromobenzyl)sulfonyl)-2-(4-hydroxy-3-nitrobenzylidene)-2H-benzo[b][1,4]thiazin-3(4H)-one; (Z)-4-((6-((2,6-difluorobenzyl)sulfonyl)-3-oxo-3,4-dihydro-2H-benzo[b][1,4]thiazin-2-ylidene)methyl)-2-nitrophenyl acetate; (Z)-4-((6-((2,6-difluorobenzyl)sulfonyl)-3-oxo-3,4-dihydro-2H-benzo[b][1,4]thiazin-2-ylidene)methyl)phenyl acetate; (Z)-2-benzylidene-6-((2,6-difluorobenzyl)sulfonyl)-2H-benzo[b][1,4]thiazin-3(4H)-one; (Z)-6-((2,6-difluorobenzyl)sulfonyl)-2-(4-methoxy-3-nitrobenzylidene)-2H-benzo[b][1,4]thiazin-3(4H)-one; (Z)-2-(3-amino-4-methoxybenzylidene)-6-((2,6-difluorobenzyl)sulfonyl)-2H-benzo[b][1,4]thiazin-3(4H)-one; (Z)-6-((2,6-difluorobenzyl)sulfonyl)-2-(4-(4-methylpiperazin-1-yl)benzylidene)-2H-benzo[b][1,4]thiazin-3(4H)-one; (Z)-6-((2,6-difluorobenzyl)sulfonyl)-2-(4-nitrobenzylidene)-2H-benzo[b][1,4]thiazin-3(4H)-one; (Z)-2-(4-aminobenzylidene)-6-((2,6-difluorobenzyl)sulfonyl)-2H-benzo[b][1,4]thiazin-3(4H)-one; (Z)-6-((2,6-difluorobenzyl)sulfonyl)-2-(4-fluorobenzylidene)-2H-benzo[b][1,4]thiazin-3(4H)-one; (Z)-2-(4-chlorobenzylidene)-6-((2,6-difluorobenzyl)sulfonyl)-2H-benzo[b][1,4]thiazin-3(4H)-one; (Z)-2-(4-bromobenzylidene)-6-((2,6-difluorobenzyl)sulfonyl)-2H-benzo[b][1,4]thiazin-3(4H)-one; (Z)-6-((2,6-difluorobenzyl)sulfonyl)-2-(4-methoxybenzylidene)-2H-benzo[b][1,4]thiazin-3(4H)-one; (Z)-6-((2,6-difluorobenzyl)sulfonyl)-2-(4-methylbenzylidene)-2H-benzo[b][1,4]thiazin-3(4H)-one, (Z)-6-((2,6-difluorobenzyl)sulfonyl)-2-(2,4,6-trimethoxybenzylidene)-2H-benzo[b][1,4]thiazin-3(4H)-one; (Z)-6-((2,6-difluorobenzyl)sulfonyl)-2-(2,4-difluorobenzylidene)-2H-benzo[b][1,4]thiazin-3(4H)-one, (Z)-6-((2,6-difluorobenzyl)sulfonyl)-2-(2,4,6-trifluorobenzylidene)-2H-benzo[b][1,4]thiazin-3(4H)-one; (Z)-methyl 2-((4-((6-((2,6-difluorobenzyl)sulfonyl)-3-oxo-3,4-dihydro-2H-benzo[b][1,4]thiazin-2-ylidene)methyl)phenyl)amino)acetate; (Z)-2-((4-((6-((2,6-difluorobenzyl)sulfonyl)-3-oxo-3,4-dihydro-2H-benzo[b][1,4]thiazin-2-ylidene)methyl)phenyl)amino)acetic acid; (Z)-6-((2,6-difluorobenzyl)sulfonyl)-2-(4-hydroxy-2,6-dimethoxybenzylidene)-2H-benzo[b][1,4]thiazin-3(4H)-one, (Z)-2-(4-chloro-3-nitrobenzylidene)-6-((2,6-difluorobenzyl)sulfonyl)-2H-benzo[b][1,4]thiazin-3(4H)-one; (Z)-6-((2,6-difluorobenzy)sulfonyl)-2-(2,4-difluorobenzylidene)-2H-benzo[b][1,4]thiazin-3(4H)-one; (Z)-6-((2,6-difluorobenzyl)sulfonyl)-2-(2,4,6-trifluorobenzylidene)-2H-benzo[b][1,4]thiazin-3(4H)-one; (Z)-methyl 4-((6-((2,6-difluorobenzyl)sulfonyl)-3-oxo-3,4-dihydro-2H-benzo[b][1,4]thiazin-2-ylidene)methyl)benzoate; (Z)-methyl 4-((6-((2,6-difluorobenzyl)sulfonyl)-3-oxo-3,4-dihydro-2H-benzo[b][1,4]thiazin-2-ylidene)methyl)-3-nitrobenzoate; (Z)-4-((6-((2,6-difluorobenzyl)sulfonyl)-3-oxo-3,4-dihydro-2H-benzo[b][1,4]thiazin-2-ylidene)methyl)-2-nitrophenyl 4-methylbenzenesulfonate; (Z)-2-(4-(2H-tetrazol-5-yl)benzylidene)-6-((2,6-difluorobenzyl)sulfonyl)-2H-benzo[b][1,4]thiazin-3(4H)-one; (Z)-2-(4-(benzyloxy)-3-nitrobenzylidene)-6-((2,6-difluorobenzyl)sulfonyl)-2H-benzo[b][1,4]thiazin-3(4H)-one; (Z)-6-((2,6-difluorobenzyl)sulfonyl)-2-(4-hydroxy-3-nitrobenzylidene)-2H-benzo[b][1,4]thiazin-3(4H)-one; (Z)-4-((6-((2,6-dimethylbenzyl)sulfonyl)-3-oxo-3,4-dihydro-2H-benzo[b][1,4]thiazin-2-ylidene)methyl)-2-nitrophenyl acetate, (Z)-4-((6-((2,6-dimethylbenzyl)sulfonyl)-3-oxo-3,4-dihydro-2H-benzo[b][1,4]thiazin-2-ylidene)methyl)phenyl acetate, (Z)-2-benzylidene-6-((2,6-dimethylbenzyl)sulfonyl)-2H-benzo[b][1,4]thiazin-3(4H)-one; (Z)-6-((2,6-dimethylbenzyl)sulfonyl)-2-(4-methoxy-3-nitrobenzylidene)-2H-benzo[b][1,4]thiazin-3(4H)-one; (Z)-2-(3-amino-4-methoxybenzylidene)-6-((2,6-dimethylbenzyl)sulfonyl)-2H-benzo[b][1,4]thiazin-3(4H)-one; (Z)-6-((2,6-dimethylbenzyl)sulfonyl)-2-(4-(4-methylpiperazin-1-yl)benzylidene)-2H-benzo[b][1,4]thiazin-3(4H)-one; (Z)-6-((2,6-dimethylbenzyl)sulfonyl)-2-(4-nitrobenzylidene)-2H-benzo[b][1,4]thiazin-3(4H)-one; (Z)-2-(4-aminobenzylidene)-6-((2,6-dimethylbenzyl)sulfonyl)-2H-benzo[b][1,4]thiazin-3(4H)-one-; (Z)-6-((2,6-dimethylbenzyl)sulfonyl)-2-(4-fluorobenzylidene)-2H-benzo[b][1,4]thiazin-3(4H)-one; (Z)-2-(4-chlorobenzylidene)-6-((2,6-dimethylbenzyl)sulfonyl)-2H-benzo[b][1,4]thiazin-3(4H)-one; (Z)-2-(4-bromobenzylidene)-6-((2,6-dimethylbenzyl)sulfonyl)-2H-benzo[b][1,4]thiazin-3(4H)-one; (Z)-6-((2,6-dimethylbenzyl)sulfonyl)-2-(4-methoxybenzylidene)-2H-benzo[b][1,4]thiazin-3(4H)-one; (Z)-6-((2,6-dimethylbenzyl)sulfonyl)-2-(4-methylbenzylidene)-2H-benzo[b][1,4]thiazin-3(4H)-one; (Z)-6-((2,6-dimethylbenzyl)sulfonyl)-2-(2,4,6-trimethoxybenzylidene)-2H-benzo[b][1,4]thiazin-3(4H)-one, (Z)-6-((2,6-dimethylbenzyl)sulfonyl)-2-(2,4-dimethylbenzylidene)-2H-benzo[b][1,4]thiazin-3(4H)-one; (Z)-6-((2,6-dimethylbenzyl)sulfonyl)-2-(2,4,6-trifluorobenzylidene)-2H-benzo[b][1,4]thiazin-3(4H)-one; (Z)-methyl 2-((4-((6-((2,6-dimethylbenzyl)sulfonyl)-3-oxo-3,4-dihydro-2H-benzo[b][1,4]thiazin-2-ylidene)methyl)phenyl)amino)acetate; (Z)-2-((4-((6-((2,6-dimethylbenzyl)sultonyl)-3-oxo-3,4-dihydro-2H-benzo[b][1,4]thiazin-2-ylidene)methyl)phenyl)amino)acetic acid; (Z)-6-((2,6-dimethylbenzyl)sulfonyl)-2-(4-hydroxy-2,6-dimethoxybenzylidene)-2H-benzo[b][1,4]thiazin-3(4H)-one; (Z)-2-(4-chloro-3-nitrobenzylidene)-6-((2,6-dimethylbenzyl)sulfonyl)-2H-benzo[b][1,4]thiazin-3(4H)-one; (Z)-6-((2,6-dimethylbenzyl)sulfonyl)-2-(2,4-difluorobenzylidene)-2H-benzo[b][1,4]thiazin-3(4H)-one; (Z)-6-((2,6-dimethylbenzyl)sulfonyl)-2-(2,4,6-trifluorobenzylidene)-2H-benzo[b][1,4]thiazin-3(4H)-one; (Z)-methyl 4-((6-((2,6-dimethylbenzyl)sulfonyl)-3-oxo-3,4-dihydro-2H-benzo[b][1,4]thiazin-2-ylidene)methyl)benzoate; (Z)-methyl 4-((6-((2,6-dimethylbenzyl)sulfonyl)-3-oxo-3,4-dihydro-2H-benzo[b][1,4]thiazin-2-ylidene)methyl)-3-nitrobenzoate; (Z)-4-((6-((2,6-dimethylbenzyl)sulfonyl)-3-oxo-3,4-dihydro-2H-benzo[b][1,4]thiazin-2-ylidene)methyl)-2-nitrophenyl 4-methylbenzenesulfonate; (Z)-2-(4-(2H-tetrazol-5-yl)benzylidene)-6-((2,6-dimethylbenzyl)sulfonyl)-2H-benzo[b][1,4]thiazin-3(4H)-one; (Z)-2-(4-(benzyloxy)-3-nitrobenzylidene)-6-((2,6-dimethylbenzyl)sulfonyl)-2H-benzo[b][1,4]thiazin-3(4H)-one; and (Z)-6-((2,6-dimethylbenzyl)sulfonyl)-2-(4-hydroxy-3-nitrobenzylidene)-2H-benzo[b][1,4]thiazin-3(4H)-one; (Z)-4-((6-((2,6-dimethoxybenzyl)sulfonyl)-3-oxo-3,4-dihydro-2H-benzo[b][1,4]thiazin-2-ylidene)methyl)-2-nitrophenyl acetate; (Z)-4-((6-((2,6-dimethoxybenzyl)sulfonyl)-3-oxo-3,4-dihydro-2H-benzo[b][1,4]thiazin-2-ylidene)methyl)phenyl acetate; (Z)-2-benzylidene-6-((2,6-dimethoxybenzyl)sulfonyl)-2H-benzo[b][1,4]thiazin-3(4H)-one; (Z)-6-((2,6-dimethoxybenzyl)sulfonyl)-2-(4-methoxy-3-nitrobenzylidene)-2H-benzo[b][1,4]thiazin-3(4H)-one; (Z)-2-(3-amino-4-methoxybenzylidene)-6-((2,6-dimethoxybenzyl)sulfonyl)-2H-benzo[b][1,4]thiazin-3(4H)-one, (Z)-6-((2,6-dimethoxybenzyl)sulfonyl)-2-(4-(4-methylpiperazin-1-yl)benzylidene)-2H-benzo[b][1,4]thiazin-3(4H)-one; (Z)-6-((2,6-dimethoxybenzyl)sulfonyl)-2-(4-nitrobenzylidene)-2H-benzo[b][1,4)thiazin-3(4H)-one; (Z)-2-(4-aminobenzylidene)-6-((2,6-dimethoxybenzyl)sulfonyl)-2H-benzo[b][1,4]thiazin-3(4H)-one; (Z)-6-((2,6-dimethoxybenzyl)sulfonyl)-2-(4-fluorobenzylidene)-2H-benzo[b][1,4]thiazin-3(4H)-one, (Z)-2-(4-chlorobenzylidene)-6-((2,6-dimethoxybenzyl) sulfonyl)-2H-benzo[b][1,4]thiazin-3(4H)-one; (Z)-2-(4-bromobenzylidene)-6-((2,6-dimethoxybenzyl)sulfonyl)-2H-benzo[b][1,4]thiazin-3(4H)-one; (Z)-6-((2,6-dimethoxybenzyl)sultonyl)-2-(4-methoxybenzylidene)-2H-benzo[b][1,4]thiazin-3(4H)-one; (Z)-6-((2,6-dimethoxybenzyl)sulfonyl)-2-(4-methylbenzylidene)-2H-benzo[b][1,4]thiazin-3(4H)-one; (Z)-6-((2,6-dimethoxybenzyl) sulfonyl)-2-(2,4,6-trimethoxybenzylidene)-2H-benzo[b][1,4]thiazin-3(4H)-one; (Z)-6-((2,6-dimethoxybenzyl)sulfonyl)-2-(2,4-dimethoxybenzylidene)-2H-benzo[b][1,4]thiazin-3(4H)-one; (Z)-6-((2,6-dimethoxybenzyl)sulfonyl)-2-(2,4,6-trifluorobenzylidene)-2H-benzo[b][1,4]thiazin-3(4H)-one, (Z)-methyl 2-((4-((6-((2,6-dimethoxybenzyl)sulfonyl)-3-oxo-3,4-dihydro-2H-benzo[b][1,4]thiazin-2-ylidene)methyl)phenyl)amino)acetate; (Z)-2-((4-((6-((2,6-dimethoxybenzyl)sulfonyl)-3-oxo-3,4-dihydro-2H-benzo[b][1,4]thiazin-2-ylidene)methyl)phenyl)amino)acetic acid; (Z)-6-((2,6-dimethoxybenzyl)sulfonyl)-2-(4-hydroxy-2,6-dimethoxybenzylidene)-2H-benzo[b][1,4]thiazin-3(4H)-one; (Z)-2-(4-chloro-3-nitrobenzylidene)-6-((2,6-dimethoxybenzyl)sulfonyl)-2H-benzo[b][1,4]thiazin-3(4H)-one; (Z)-6-((2,6-dimethoxybenzyl)sulfonyl)-2-(2,4-difluorobenzylidene)-2H-benzo[b][1,4]thiazin-3(4H)-one; (Z)-6-((2,6-dimethoxybenzyl)sultonyl)-2-(2, 4,6-trifluorobenzylidene)-2H-benzo[b][1,4]thiazin-3(4H)-one; (Z)-methyl 4-((6-((2,6-dimethoxybenzyl)sulfonyl)-3-oxo-3,4-dihydro-2H-benzo[b][1,4]thiazin-2-ylidene)methyl)benzoate; (Z)-methyl 4-((6-((2,6-dimethoxybenzyl)sulfonyl)-3-oxo-3,4-dihydro-2H-benzo[b][1,4]thiazin-2-ylidene)methyl)-3-nitrobenzoate; (Z)-4-((6-((2,6-dimethoxybenzyl)sulfonyl)-3-oxo-3,4-dihydro-2H-benzo[b][1,4]thiazin-2-ylidene)methyl)-2-nitrophenyl 4-methylbenzenesulfonate; (Z)-2-(4-(2H-tetrazol-5-yl)benzylidene)-6-((2,6-dimethoxybenzyl)sulfonyl)-2H-benzo[b][1,4]thiazin-3(4H)-one; (Z)-2-(4-(benzyloxy)-3-nitrobenzylidene)-6-((2,6-dimethoxybenzyl)sulfonyl)-2H-benzo[b][1,4]thiazin-3(4H)-one; and (Z)-6-((2,6-dimethoxybenzyl)sulfonyl)-2-(4-hydroxy-3-nitrobenzylidene)-2H-benzo[b][1,4]thiazin-3(4H)-one

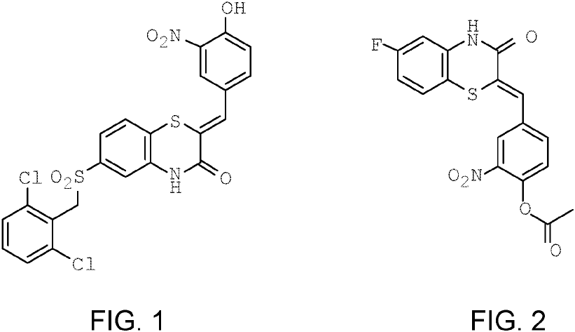

In a specific embodiment, a compound is a compound described in U.S. Pat No. 9,242,945 and International Patent Application Publication No. WO 2012/166586 A2, each of which is incorporated herein by reference in its entirety. In another specific embodiment, a compound described herein is 108110 (FIG. 2). In another specific embodiment, a compound described herein is 108600 (FIG. 1).

A compound described herein may be produced using any technique known to one of skill (such as, e.g., described in U.S. Pat No. 9,242,945 and International Patent Application Publication No. WO 2012/166586 A2, each of which is incorporated herein by reference in its entirety).

In the following paragraphs some of the definitions include examples. The examples are intended to be illustrative, and not limiting.

The term “alkyl”, by itself or as part of another substituent means, unless otherwise stated, a straight, branched (chiral or achiral) or cyclic chain hydrocarbon having the number of carbon atoms designated (e.g. C₁-C₆ means one to six carbons) and includes straight, branched chain or cyclic groups. Examples include: methyl, ethyl, propyl, isopropyl, butyl, isobutyl, tert-butyl, pentyl, neopentyl, hexyl, cyclohexyl and cyclopropylmethyl. Most preferred is (C₁-C₃)alkyl, particularly ethyl, methyl and isopropyl.

The term “alkenyl” employed alone or in combination with other terms, means, unless otherwise stated, a stable mono-unsaturated or di-unsaturated straight chain, branched chain or cyclic hydrocarbon group having the stated number of carbon atoms. Examples include vinyl, propenyl (allyl), crotyl, isopentenyl, butadienyl, 1,3-pentadienyl, 1,4-pentadienyl, cyclopentenyl, cyclopentadienyl and the higher homologs and isomers. A functional group representing an alkene is exemplified by —CH═CH—CH₂—.

The term “alkynyl” employed alone or in combination with other terms, means, unless otherwise stated, a straight chain or branched non-cyclic hydrocarbon having the stated number of carbon atoms (e.g., 2 to 8 carbon atoms) and including at lease one carbon-carbon triple bond. Representative straight chain and branched -(C₂-C₈)alkynyls include -acetylenyl, -propynyl, -1-butynyl, -2-butynyl, -1-pentynyl, -2-pentynyl, -3-methyl-1-butynyl, -4-pentynyl, -1-hexynyl, -2-hexynyl, -5-hexynyl, -1-heptynyl, -2-heptynyl, -6-heptynyl, -1-octynyl, -2-octynyl, -7-octynyl, and the like.

The term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. Preferred are (C₁-C₃)alkoxy, particularly ethoxy and methoxy.

The terms “halo” or “halogen” by themselves or as part of another substituent mean, unless otherwise stated, a fluorine, chlorine, bromine, or iodine atom, preferably, fluorine, chlorine, or bromine, more preferably, fluorine or chlorine.

The term “(Cₓ-C_{y})perfluoroalkyl,” wherein x<y, means an alkyl group with a minimum of x carbon atoms and a maximum of y carbon atoms, wherein all hydrogen atoms are replaced by fluorine atoms. Preferred is —(C₁-C₆)perfluoroalkyl, more preferred is —(C₁-C₃)perfluoroalkyl, most preferred is —CF₃.

The term “(Cₓ-C_{y})perfluoroalkoxy,” wherein x<y, means an alkoxy group with a minimum of x carbon atoms and a maximum of y carbon atoms, wherein all hydrogen atoms are replaced by fluorine atoms. Preferred is —(C₁-C₆)perfluoroalkoxy, more preferred is —(C₁-C₃)perfluoroalkoxy, most preferred is —OCF₃.

The term “aromatic” refers to a carbocycle or heterocycle having one or more polyunsaturated rings having aromatic character (i.e. having (4n+2) delocalized π (pi) electrons where n is an integer).

The term “aryl” employed alone or in combination with other terms, means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl; anthracyl; and naphthyl. Preferred are phenyl and naphthyl, most preferred is phenyl.

The term “optionally substituted aryl-(C₁-C₃)alkyl” means a functional group wherein a one to three carbon alkylene chain is attached to an optionally substituted aryl group, e.g., -CH₂CH₂-phenyl. Similarly, the term “optionally substituted heteroaryl(C₁-C₃)alkyl” means a functional group wherein a one to three carbon alkylene chain is attached to an optionally substituted heteroaryl group, e.g., —CH₂CH₂-pyridyl.

The term “heterocycle” or “heterocyclyl” or “heterocyclic” by itself or as part of another substituent means, unless otherwise stated, an unsubstituted or substituted, stable, nonaromatic mono- or multi-cyclic ring system which consists of carbon atoms and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom which affords a stable structure.

The term “heteroaryl” or “heteroaromatic” refers to an unsubstituted or substituted, stable, mono- or multi-cyclic ring system having aromatic character which consists of carbon atoms and at least one heteroatom selected from the group consisting of N, O, and S, and wherein the nitrogen and sulfur heteroatoms may be optionally oxidized, and the nitrogen atom may be optionally quaternized. The heteroaryl or heteroaromatic system may be attached, unless otherwise stated, at any heteroatom or carbon atom which affords a stable structure.

A polycyclic heteroaryl may include one or more rings which are partially saturated. Examples include tetrahydroquinoline and 2,3-dihydrobenzofuryl.

Examples of heterocycles include monocyclic groups such as: aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin and hexamethyleneoxide.

Examples of heteroaryl groups include: pyridyl, pyrazinyl, pyrimidinyl, particularly 2-and 4-pyrimidinyl, pyridazinyl, thienyl, furyl (furanyl), pyrrolyl, particularly 2-pyrrolyl, imidazolyl, thiazolyl, oxazolyl, pyrazolyl, particularly 3- and 5-pyrazolyl, isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heteroaryls include: indolyl, particularly 3-, 4-, 5-, 6- and 7-indolyl, indolinyl, quinolyl, tetrahydroquinolyl, isoquinolyl, particularly 1- and 5-isoquinolyl, 1,2,3,4-tetrahydroisoquinolyl, cinnolinyl, quinoxalinyl, particularly 2- and 5-quinoxalinyl, quinazolinyl, phthalazinyl, 1,8-naphthyridinyl, 1,4-benzodioxanyl, coumarinyl, dihydrocoumarinyl, benzofuryl, 1,5-naphthyridinyl, 2,3-dihydrobenzofuryl, 1,2-benzisoxazolyl, benzothienyl, particularly 3-, 4-, 5-, 6-, and 7-benzothienyl, benzoxazolyl, benzthiazolyl, particularly 2-benzothiazolyl and 5-benzothiazolyl, purinyl, benzimidazolyl, particularly 2-benzimidazolyl, benztriazolyl, thioxanthinyl, carbazolyl, carbolinyl, acridinyl, pyrrolizidinyl, and quinolizidinyl.

The aforementioned listing of heterocyclyl and heteroaryl moieties is intended to be representative and not limiting.

For compounds of the present invention, when an aromatic or heteroaromatic ring is attached to a position and the ring comprises a polycyclic ring which is partially saturated, the attachment point on the aromatic or heteroaromatic ring is on a ring atom of an aromatic ring component of the polycyclic ring. For example on the partially saturated heteroaromatic ring, 1,2,3,4-tetrahydroisoquinoline, attachment points would be ring atoms at the 5-, 6-, 7- and 8-positions.

The phrase “optionally substituted” means that an atom or group of atoms has optionally replaced hydrogen as the substituent attached to another group. For aryl and heteroaryl groups, the term “optionally substituted” refers to any level of optional substitution, namely mono-, di-, tri-, tetra-, or penta-substitution, where such substitution(s) are permitted. When present, the substituents are independently selected, and each optional substitution may be at any chemically accessible position.

### 4.2 Compositions

Provided herein are compositions (e.g., pharmaceutical compositions) comprising a compound described herein having the desired degree of purity in a pharmaceutically acceptable carrier, excipient or stabilizer (Remington’s Pharmaceutical Sciences (1990) Mack Publishing Co., Easton, PA). In a specific embodiment, a composition comprises a compound described herein and an acceptable carrier or excipient. Examples of pharmaceutical compositions (or preparations) of a compound described herein that may be used are described in U.S. Pat. No. 9,242,945 and International Patent Application Publication No. WO 2012/166586 A2.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations can be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN®80). A sequestering or chelating agent of metal ions includes EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

A pharmaceutical composition may be formulated for any route of administration to a subject. Specific examples of routes of administration include oral, intransal, transdermal, intradermal, parenteral, and mucosal. In a specific embodiment, the composition is formulated for oral administration. In another specific embodiment, the composition is formulated for intramuscular or intravenous administration. Parenteral administration, characterized by either subcutaneous, intramuscular or intravenous injection, is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol.

In certain embodiments, a compound described herein is administered in a tablet, capsule or other oral formulation. In some embodiments, a compound described herein is administered in a pharmaceutical composition known to one of skill in the art. A pharmaceutical composition comprising a compound described herein may be used to treat a virus (e.g., an RNA virus) infection or disease associated therewith. A pharmaceutical composition comprising a compound described herein may also be used to prevent a disease associated with a virus (e.g., an RNA virus) infection. In a specific embodiment, a pharmaceutical composition comprising a compound described herein is used to treat a SARS-CoV-2 infection or COVID-19. In another specific embodiment, a pharmaceutical composition comprising a compound described herein is used to prevent COVID-19.

### 4.3 Prophylactic and Therapeutic Uses

In one aspect, provided herein are methods for treating a virus infection or disease associated therewith comprising administering a compound described herein to a subject in need thereof. In a specific embodiment, provided herein is a method for treating a virus infection or disease associated therewith in a subject comprising administering to the subject an effective amount of a compound described herein. In another specific embodiment, provided herein is a method for treating a virus infection or disease associated therewith in a subject comprising administering to the subject a pharmaceutical composition comprising an effective amount of a compound described herein. In another specific embodiment, provided herein is a method for treating a virus infection or disease associated therewith comprising administering to the subject an effective amount of a compound described herein and another therapy, such as known to one of skill in the art or described herein (see, e.g., Section 4.3.2). In another specific embodiment, provided herein is a method for treating a virus infection or disease associated therewith in a subject comprising administering to the subject a pharmaceutical composition comprising an effective amount of a compound described herein, and another therapy, such as known to one of skill in the art or described herein (see, e.g., Section 4.3.2). In a specific embodiment, the compound is a compound of Formula (I). In another specific embodiment, the compound is 108600. In another specific embodiment, the compound is 108110.

In another aspect, provided herein are methods for treating an RNA virus infection or a disease associated therewith comprising administering a compound described herein to a subject in need thereof. In a specific embodiment, provided herein is a method for treating an RNA virus infection or a disease associated therewith in a subject comprising administering to the subject an effective amount of a compound described herein. In another specific embodiment, provided herein is a method for treating an RNA virus infection or a disease associated therewith in a subject comprising administering to the subject a pharmaceutical composition comprising an effective amount of a compound described herein . In another specific embodiment, provided herein is a method for treating an RNA virus infection or a disease associated therewith comprising administering to the subject an effective amount of a compound described herein and another therapy, such as known to one of skill in the art or described herein (see, e.g., Section 4.3.2). In another specific embodiment, provided herein is a method for treating an RNA virus infection or disease associated therewith in a subject comprising administering to the subject a pharmaceutical composition comprising an effective amount of a compound described herein, and another therapy, such as known to one of skill in the art or described herein (see, e.g., Section 4.3.2). In a specific embodiment, the compound is a compound of Formula (I). In another specific embodiment, the compound is 108600. In another specific embodiment, the compound is 108110.

In another aspect, provided herein are methods for preventing a disease associated with a virus infection comprising administering a compound described herein to a subject in need thereof. In a specific embodiment, provided herein is a method for preventing a disease associated with a virus infection in a subject comprising administering to the subject an effective amount of a compound described herein. In another specific embodiment, provided herein is a method for preventing a disease associated with a virus infection in a subject comprising administering to the subject a pharmaceutical composition comprising an effective amount of a compound described herein . In another specific embodiment, provided herein is a method for preventing a disease associated with a virus infection comprising administering to the subject an effective amount of a compound described herein and another therapy, such as known to one of skill in the art or described herein (see, e.g., Section 4.3.2). In another specific embodiment, provided herein is a method for preventing a disease associated with a virus infection in a subject comprising administering to the subject a pharmaceutical composition comprising an effective amount of a compound described herein, and another therapy, such as known to one of skill in the art or described herein (see, e.g., Section 4.3.2). In a specific embodiment, the compound is a compound of Formula (I). In another specific embodiment, the compound is 108600. In another specific embodiment, the compound is 108110.

In another aspect, provided herein are methods for preventing a disease associated with an RNA virus infection comprising administering a compound described herein to a subject in need thereof. In a specific embodiment, provided herein is a method for preventing a disease associated with an RNA virus infection in a subject comprising administering to the subject an effective amount of a compound described herein. In another specific embodiment, provided herein is a method for preventing a disease associated with an RNA virus infection in a subject comprising administering to the subject a pharmaceutical composition comprising an effective amount of a compound described herein. In another specific embodiment, provided herein is a method for preventing a disease associated with an RNA virus infection comprising administering to the subject an effective amount of a compound described herein and another therapy, such as known to one of skill in the art or described herein (see, e.g., Section 4.3.2). In another specific embodiment, provided herein is a method for preventing a disease associated with an RNA virus infection in a subject comprising administering to the subject a pharmaceutical composition comprising an effective amount of a compound described herein, and another therapy, such as known to one of skill in the art or described herein (see, e.g., Section 4.3.2). In a specific embodiment, the compound is a compound of Formula (I). In another specific embodiment, the compound is 108600. In another specific embodiment, the compound is 108110.

The RNA virus may be single stranded or double-stranded, positive or negative sense, and segmented or non-segmented. In some embodiments, the RNA virus is a single-stranded, positive sense RNA virus. In other embodiments, the RNA virus is a single-stranded, negative sense segmented or non-segmented virus. In a specific embodiment, the RNA virus is a coronavirus (e.g., SARS-CoV-1 or SARS-CoV-2), an influenza virus (e.g., an influena A virus or an influenza B virus), a heptatitis C virus, a vesicular stomatitis virus (VSV) or a Newcastle disease virus (NDV).

In a particular embodiment, the administration of an effective amount of a compound described herein to the subject inhibits or reduces in the progression of a disease associated with a virus (e.g., an RNA virus). In another embodiment, the administration of an effective amount of a compound described herein to the subject inhibits or reduces onset, development and/or severity of a symptom (e.g., fever, myalgia, cough, difficulty breathing, tiredness) of a virus (e.g., an RNA virus) infection or disease associated therewith. In another embodiment, the administration of an effective amount of a compound described herein to the subject inhibits or reduces duration of a virus (e.g., an RNA virus) infection, or a disease or a symptom associated therewith. In another embodiment, the administration of an effective amount of a compound described herein to the subject reduces organ failure associated with a virus (e.g., an RNA virus) infection or a disease associated therewith. In another embodiment, the administration of an effective amount of a compound described herein to the subject reduces the hospitalization of the subject. In another embodiment, the administration of an effective amount of a compound described herein to the subject reduces the length of hospitalization of the subject. In another embodiment, the administration of an effective amount of a compound described herein to the subject increases the overall survival of subjects with a virus (e.g., an RNA virus) infection or a disease associated therewith. In another embodiment, the administration of an effective amount of a compound described herein to the subject prevents the onset or progression of a secondary infection associated with virus (e.g., RNA virus) infection.

In a specific embodiment, administration of a compound described herein to a subject reduces the incidence of hospitalization by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to the incidence of hospitalization in the absence of administration of a compound described herein.

In a specific embodiment, administration of a compound described herein to a subject reduces mortality by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to the mortality in the absence of administration of a compound described herein.

In certain embodiments, the administration of an effective amount of a compound described herein to a subject results in one, two, three, four, five, or more of the following effects: (i) reduction or amelioration in the severity of a virus (e.g., an RNA virus) infection, or a disease or a symptom associated therewith; (ii) reduction in the duration of a virus (e.g., an RNA virus) infection, or a disease or a symptom associated therewith; (iii) prevention of the progression of a virus (e.g., an RNA virus) infection, or a disease or a symptom associated therewith; (iv) regression of a virus (e.g., an RNA virus) infection, or a disease or a symptom associated therewith; (v) prevention of the development or onset of a symptom of a virus (e.g., an RNA virus) infection or a disease associated therewith; (vi) reduction in organ failure associated a virus (e.g., an RNA virus) infection or a disease associated therewith; (vii) reduction in the hospitalization of a subject; (viii) reduction in the hospitalization length; (ix) an increase in the survival of a subject with a virus (e.g., an RNA virus) infection or a disease associated therewith; (x) reduction in virus (e.g,. RNA virus) titer; (xi) the reduction in the number of symptoms associated with a virus (e.g., an RNA virus) infection or a disease associated therewith; (xiii) inhibition of replication of the virus (e.g., RNA virus); (xiv) enhancement, improvement, supplementation, complementation, or augmentation of the prophylactic or therapeutic effect(s) of another therapy; (xii) prevention of the onset or progression of a secondary infection associated with a virus (e.g., RNA virus) infection; and/or (xiii) prevention of the onset or diminution of disease severity of bacterial infection occurring secondary to a virus (e.g., RNA virus) infection.

In a specific aspect, provided herein are methods for treating a SARS-CoV-2 infection or COVID-19 comprising administering a compound described herein to a subject in need thereof. In a specific embodiment, provided herein is a method for treating a SARS-CoV-2 infection or COVID-19 in a subject comprising administering to the subject an effective amount of a compound described herein. In another specific embodiment, provided herein is a method for treating a SARS-CoV-2 infection or COVD-19 in a subject comprising administering to the subject a pharmaceutical composition comprising an effective amount of a compound described herein . In another specific embodiment, provided herein is a method for treating a SARS-CoV-2 infection or COVID-19 comprising administering to the subject an effective amount of a compound described herein and another therapy, such as known to one of skill in the art or described herein (see, e.g., Section 4.3.2). In another specific embodiment, provided herein is a method for treating a SARS-CoV-2 infection or COVID-19 in a subject comprising administering to the subject a pharmaceutical composition comprising an effective amount of a compound described herein, and another therapy, such as known to one of skill in the art or described herein (see, e.g., Section 4.3.2). In a specific embodiment, the compound is a compound of Formula (I). In another specific embodiment, the compound is 108600. In another specific embodiment, the compound is 108110.

In another specific aspect, provided herein are methods for preventing COVID-19 comprising administering a compound described herein to a subject in need thereof. In a specific embodiment, provided herein is a method for preventing COVID-19 in a subject comprising administering to the subject an effective amount of a compound described herein . In another specific embodiment, provided herein is a method for preventing COVID-19 in a subject comprising administering to the subject a pharmaceutical composition comprising an effective amount of a compound described herein. In another specific embodiment, provided herein is a method for preventing COVID-19 comprising administering to the subject an effective amount of a compound described herein and another therapy, such as known to one of skill in the art or described herein (see, e.g., Section 4.3.2). In another specific embodiment, provided herein is a method for preventing COVID-19 in a subject comprising administering to the subject a pharmaceutical composition comprising an effective amount of a compound described herein, and another therapy, such as known to one of skill in the art or described herein (see, e.g., Section 4.3.2). In a specific embodiment, the compound is a compound of Formula (I). In another specific embodiment, the compound is 108600. In another specific embodiment, the compound is 108110.

In a particular embodiment, the administration of an effective amount of a compound described herein to the subject inhibits or reduces in the progression of COVID-19. In another embodiment, the administration of an effective amount of a compound described herein to the subject inhibits or reduces onset, development and/or severity of a symptom (e.g., fever, myalgia, cough, difficulty breathing, tiredness) of COVID-19. In another embodiment, the administration of an effective amount of a compound described herein to the subject inhibits or reduces duration of COVID-19 or a symptom associated therewith. In another embodiment, the administration of an effective amount of a compound described herein to the subject reduces organ failure associated with COVID-19. In another embodiment, the administration of an effective amount of a compound described herein to the subject reduces the hospitalization of the subject. In another embodiment, the administration of an effective amount of a compound described herein to the subject reduces the length of hospitalization of the subject. In another embodiment, the administration of an effective amount of a compound described herein to the subject increases the overall survival of subjects with COVID-19. In another embodiment, the administration of an effective amount of a compound described herein to the subject prevents the onset or progression of a secondary infection associated with SARS-CoV-2 infection.

In a specific embodiment, administration of a compound described herein to a subject reduces the incidence of hospitalization by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to the incidence of hospitalization in the absence of administration of a compound described herein .

In a specific embodiment, administration of a compound described herein to a subject reduces mortality by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to the mortality in the absence of administration of a compound described herein.

In certain embodiments, the administration of an effective amount of a compound described herein to a subject results in one, two, three, four, five, or more of the following effects: (i) reduction or amelioration in the severity of a SARS-CoV-2 infection, COVID-19 or a symptom associated therewith; (ii) reduction in the duration of a SARS-CoV-2 infection, COVID-19 or a symptom associated therewith; (iii) prevention of the progression of a SARS-CoV-2 infection, COVID-19 or a symptom associated therewith; (iv) regression of a SARS-CoV-2 infection, COVID-19 or a symptom associated therewith; (v) prevention of the development or onset of a symptom of a SARS-CoV-2 infection or COVID-19; (vi) reduction in organ failure associated with a SARS-CoV-2 infection or COVID-19; (vii) reduction in the hospitalization of a subject; (viii) reduction in the hospitalization length; (ix) an increase in the survival of a subject with a SARS-CoV-2 infection or COVID-19; (x) reduction in SARS-CoV-2 titer; (xi) the reduction in the number of symptoms associated with a SARS-CoV-2 infection or COVID-19; (xxiii) enhancement, improvement, supplementation, complementation, or augmentation of the prophylactic or therapeutic effect(s) of another therapy; (xii) prevention of the onset or progression of a secondary infection associated with a SARS-CoV-2 infection or COVID-19; and/or (xiii) prevention of the onset or diminution of disease severity of bacterial pneumonias occurring secondary to a SARS-CoV-2 infection or COVID-19.

In a specific embodiment, administration of a compound described herein to a subject reduces the number of and/or the frequency of symptoms of in the subject (exemplary symptoms of a SARS-CoV-2 include, but are not limited to, body aches (especially joints and throat), fever, nausea, headaches, fatigue, sore throat, and difficulty breathing). In another specific embodiment, administration of a compound described herein to a subject reduces the progression of a SARS-CoV-2 infection or COVID-19 using the WHO ordinal scale. In another specific embodiment, administration of a compound described herein to a subject reduces the need for invasive mechanical ventilation. In another specific embodiment, administration of a compound described herein to a subject reduces the need to provide oxygen supplementation to the subject. In another specific embodiment, administration of a compound described herein to a subject reduces the mortality caused by a SARS-CoV-2 infection or COVID-19.

The ability of a compound described herein to inhibit or reduce replication of a virus (e.g., an RNA virus) is assessed using in an assay described in Section 5, infra. In a specific embodiment, a compound that inhibits or reduces replication of a virus (e.g., an RNA virus) in such an assay has utility in the treatment of a virus (e.g., an RNA virus) infection or a disease associated therewith. In another specific embodiment, a compound that inhibits or reduces replication of a virus (e.g., an RNA virus) in such an assay has utility in the prevention of a disease associated with a virus (e.g., an RNA virus) infection.

A compound described herein may be administered alone or in combination with another/other type of therapy known in the art. See, e.g., Section 4.3.2 for other therapies.

In specific embodiment, a compound described herein may be used as any line of therapy, including, but not limited to, a first, second, third, fourth and/or fifth line of therapy.

### 4.3.1 Routes of Administration and Dosage

A compound described herein or composition described herein may be delivered to a subject by a variety of routes. These include, but are not limited to, oral, intradermal, intramuscular, intraperitoneal, transdermal, intravenous, intranasal and subcutaneous routes. In a specific embodiment, a route known to one of skill in the art is used to administer a compound described herein or composition thereof.

Exemplary dosages of a compound described herein are 280 mg 560 mg, 840 mg, and 1120 mg. In a specific embodiment, a dosage of a compound described herein or composition thereof known to one of skill in the art is used to treat a subject in accordance with the methods described herein. An exemplary treatment regime entails administration once or twice per day for a period of 3 days, 5 days, 7 days, 14 days, 28 days, 2 months, 3 months, or more. In another specific embodiment, administration of a compound described herein to a subject is discontinued if the subject experiences an adverse event.

### 4.3.2 Combination Therapy

In various embodiments, a compound described herein or composition described herein may be administered to a subject in combination with one or more other therapies (e.g., antiviral or immunomodulatory therapies). In some embodiments, a pharmaceutical composition described herein may be administered to a subject in combination with one or more therapies. The one or more other therapies may be in the same composition or a different composition as a compound described herein .

In some embodiments, the one or more other therapies that are supportive measures, such as pain relievers, anti-fever medications, or therapies that alleviate or assist with breathing. Specific examples of supportive measures include humidification of the air by an ultrasonic nebulizer, aerolized racemic epinephrine, oral dexamethasone, intravenous fluids, intubation, fever reducers (e.g., ibuprofen or acetometaphin), and antibiotic and/or antifungal therapy (i.e., to prevent or treat secondary bacterial and/or fungal infections).

In certain embodiments, the therapies are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In some embodiments, two or more therapies are administered concurrently. The two or more therapies can be administered in the same composition or a different composition. Further, the two or more therapies can be administered by the same route of administration of a different route of administration.

### 4.3.3 Patient Populations

As used herein, the terms “subject” and “patient” are used interchangeably to refer to an animal (e.g., birds, reptiles, and mammals, such as humans). In one embodiment, a patient treated in accordance with the methods provided herein is a patient suffering from or expected to suffer from a virus (e.g., RNA virus) infection or a disease associated therewith. In another embodiment, a patient treated in accordance with the methods provided herein is a patient exposed to a virus (e.g., RNA virus) infection but not manifesting any symptoms of the infection or a disease associated therewith. In another embodiment, a patient treated in accordance with the methods provided herein is a patient experiencing one or more symptoms of a virus (e.g., RNA virus) infection or a disease associated therewith. In another embodiment, a patient treated in accordance with the methods provided herein is a patient diagnosed with a virus (e.g., RNA virus) infection or a disease associated therewith. In some embodiments, a patient treated in accordance with the methods provided herein is a patient infected with a virus (e.g., RNA virus) that does not manifest any symptoms of the infection or a disease associated therewith. In certain embodiments, a patient treated in accordance with the methods provided herein is a patient infected with a virus (e.g., RNA virus) that manifests mild symptoms of the infection or a disease associated therewith. In some embodiments, a patient treated in accordance with the methods provided herein is a patient infected with a virus (e.g., RNA virus) that manifests moderate symptoms of the infection or a disease associated therewith. In certain embodiments, a patient treated in accordance with the methods provided herein is a patient infected with a virus (e.g., RNA virus) that manifests moderate to severe symptoms of the infection or a disease associated therewith.

In another embodiment, a patient treated in accordance with the methods provided herein is a patient suffering from or expected to suffer from a SARS-CoV-2 infection or COVID-19. In another embodiment, a patient treated in accordance with the methods provided herein is a patient exposed to a SARS-CoV-2 infection but not manifesting any symptoms of the infection or COVID-19. In another embodiment, a patient treated in accordance with the methods provided herein is a patient diagnosed with a SARS-CoV-2 infection or COVID-19. In some embodiments, a patient treated in accordance with the methods provided herein is a patient infected with a SARS-CoV-2 that does not manifest any symptoms of the infection or COVID-19. In certain embodiments, a patient treated in accordance with the methods provided herein is a patient infected with a SARS-CoV-2 that manifests mild symptoms of the infection or COVID-19. In some embodiments, a patient treated in accordance with the methods provided herein is a patient infected with a SARS-CoV-2 that manifests moderate symptoms of the infection or COVID-19. In certain embodiments, a patient treated in accordance with the methods provided herein is a patient infected with a SARS-CoV-2 that manifests moderate to severe symptoms of the infection or COVID-19.

In another embodiment, a patient treated in accordance with the methods provided herein is a patient experiencing one or more symptoms of COVID-19. Symptoms of COVID-19 include, but are not limited to, body aches (especially joints and throat), fever, nausea, headaches, cough, fatigue, sore throat, lack of smell, lack of taste, congestion, diarrhea, and difficulty breathing. In another embodiment, a patient treated in accordance with the methods provided herein is a patient with COVID-19 who does not manifest symptoms of the disease that are severe enough to require hospitalization. In another embodiment, a patient treated in accordance with the methods provided herein is a patient with COVID-19 manifesting symptoms of the disease that are severe enough to require hospitalization.

In a specific embodiment, a patient treated in accordance with the methods provided herein is a human. In certain embodiments, a patient treated in accordance with the methods provided herein is a human infant. In some embodiments, a patient treated in accordance with the methods provided herein is a human toddler. In certain embodiments, a patient treated in accordance with the methods provided herein is a human child. In other embodiments, a patient treated in accordance with the methods provided herein is a human adult. In some embodiments, a patient treated in accordance with the methods provided herein is an elderly human. In certain embodiments, a patient treated in accordance with the methods provided herein is patient that is pregnant. As used herein, the term “human adult” refers to a human that is 18 years or older. As used herein, the term “human child” refers to a human that is 1 year to 18 years old. As used herein, the term “human infant” refers to a newborn to 1 year old human. As used herein, the term “human toddler” refers to a human that is 1 years to 3 years old. As used herein, the term “elderly human” refers to a human that is 65 years old and older.

In some embodiments, a patient treated in accordance with the methods provided herein is a patient infected by a virus (e.g., an RNA virus, such as SARS-CoV-2) with a condition that increases susceptibility to virus (e.g., the RNA virus, such as SARS-CoV-2) complications or for which the virus (e.g., the RNA virus, such as SARS-CoV-2) increases complications associated with the condition such as, e.g., conditions that affect the lung, such as cystic fibrosis, asthma, chronic obstructive pulmonary disease, emphysema, or bacterial infections; cardiovascular disease; or diabetes. Other conditions that may increase virus (e.g., an RNA virus, such as SARS-CoV-2) complications include kidney disorders; blood disorders (including anemia or sickle cell disease); or weakened immune systems (including immunosuppression caused by medications, malignancies such as cancer, organ transplant, or HIV infection). In some embodiments, a patient treated in accordance with the methods provided herein is any subject with a virus (e.g., an RNA virus, such as SARS-CoV-2) infection who is immunocompromised or immunodeficient.

In certain embodiments, patients treated in accordance with the methods provided herein are patients already being treated with antibiotics, antivirals, antifungals, or other biological therapy/immunotherapy.

### 4.4 Kits

In another aspect, provided herein is a pharmaceutical pack or kit comprising one or more containers filled with a composition (e.g., a pharmaceutical compositions) described herein. In a specific embodiment, provided herein is a pharmaceutical pack or kit comprising one or more containers filled with a compound described herein . Optionally associated with such container(s) can be a notice in the form prescribed by a governmental agency regulating the manufacture, use or sale of pharmaceuticals or biological products, which notice reflects approval by the agency of manufacture, use or sale for human administration. The kits encompassed herein can be used in the above methods.

### EXAMPLE 1: INHIBITION OF SARS-COV-2 REPLICATION

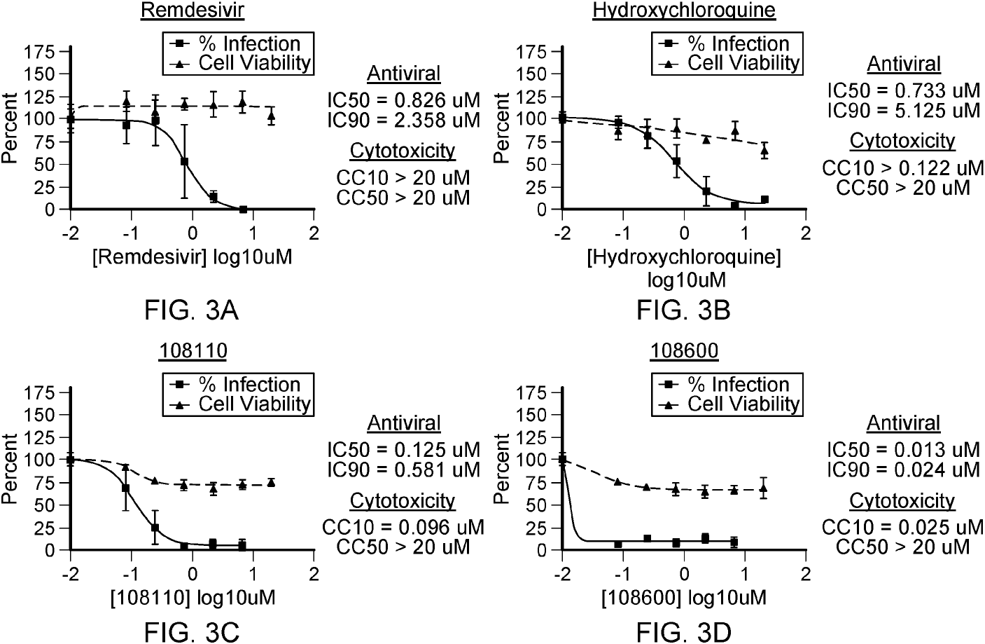

Methods: 2,000 Vero E6 cells were seeded into 96-well plates in DMEM (10% FBS) and incubated for 24 h at 37C, 5% CO₂. Two hours before infection, the medium was replaced with 100 µl of DMEM (2% FBS) containing the compound of interest at concentrations 50% greater than those indicated, including a DMSO control. Plates were then transferred into the Biosafety Level 3 (BSL3) facility and 100 PFU (MOI 0.025) was added in 50 µl of DMEM (2% FBS), bringing the final compound concentration to those indicated in FIGS. 3A-3D. Plates were then incubated for 48 h at 37° C. After infection, supernatants were removed and cells were fixed with 4% formaldehyde for 24 hours prior to being removed from the BSL3 facility. The cells were then immunostained for the viral NP protein with a DAPI counterstain. Infected cells (488 nM) and total cells (DAPI) were quantified using the Celigo (Nexcelcom) imaging cytometer. Percent infection was quantified as ((Infected cells/Total cells) - Background) *100 and the DMSO control was then set to 100% infection for analysis. The IC₅₀ and IC₉₀ for each experiment were determined using the Prism (GraphPad Software) software. For select inhibitors, infected supernatants were assayed for infectious viral titer using the TCID₅₀ method. Cytotoxicity was also performed using the MTT assay (Roche), according to the manufacturer’s instructions. Cytotoxicity was performed in uninfected VeroE6 cells with same compound dilutions and concurrent with viral replication assay.

TCID₅₀ Assay: Infectious supernatants were collected at 48h post infection and frozen at -80° C. until later use. Infectious titers were quantified by limiting dilution titration on Vero E6 cells. Briefly, Vero E6 cells were seeded in 96-well plates at 20,000 cells/well. The next day, SARS-CoV2-containing supernatant was applied at serial 10-fold dilutions ranging from 10⁻¹ to 10⁻⁶ and, after 5 days, viral CPE was detected by staining cell monolayers with crystal violet. Median tissue culture infectious doses (TCID₅₀)/mL were calculated using the method of Reed and Muench (L.J. Reed, H. Muench. A simple method of estimating fifty percent endpoint. Am. J. Hyg., 27 (1938), p. 493).

Results: The results of the study are shown in the FIGS. 3A-3D given below.

In this study, 2,000 Vero E6 cells were seeded into 96-well plates in DMEM (10% FBS) and incubated for 24 hrs at 37° C., in the presence of 5% CO₂. Vero E6 cells used were purchased from ATCC and thus authenticated (VERO C1008 [Vero 76, clone E6, Vero E6] (ATCC®CRL-1586™); tested negative for mycoplasma contamination prior to commencement). Two hours before infection, the medium was replaced with 100 µl of DMEM (2% FBS) containing the compound of interest at concentrations 50% greater than those indicated in FIGS. 3A-3D, including a DMSO control. Plates were then transferred into the BSL3 facility and 100 PFU (MOI 0.025) was added in 50 µl of DMEM (2% FBS), bringing the final compound concentration to those indicated in FIGS. 3A-3D. Plates were then incubated for 48 hrs at 37° C. After infection, supernatants were removed and cells were fixed with 4% formaldehyde for 24 hours prior to being removed from the BSL3 facility. The cells were then immunostained for the viral NP protein (antisera produced in the Garcia-Sastre lab; 1:10,000) with a DAPI counterstain. Infected cells (488 nM) and total cells (DAPI) were quantified using the Celigo (Nexcelcom) imaging cytometer. Infectivity is measured by the accumulation of viral NP protein in the nucleus of the Vero E6 cells (fluorescence accumulation). Percent infection was quantified as ((Infected cells/Total cells) -Background) * 100 and the DMSO control was then set to 100% infection for analysis. The IC₅₀ and IC₉₀ for each experiment were determined using the Prism (Graph Pad Software) software. For select inhibitors, infected supernatants were assayed for infectious viral titer using the Median Tissue Culture Infectious Dose (TCID)₅₀ method. For this, infectious supernatants were collected at 48h post infection and frozen at -80° C. until later use. Infectious titers were quantified by limiting dilution titration on Vero E6 cells. Briefly, Vero E6 cells were seeded in 96-well plates at 20,000 cells/well. The next day, SARS-CoV-2-containing supernatant was applied at serial 10-fold dilutions ranging from 10⁻¹ to 10⁻⁶ and, after 5 days, viral CPE was detected by staining cell monolayers with crystal violet. TCID₅₀/mL were calculated using the method of Reed and Muench. Cytotoxicity was also performed using the MTT assay (Roche), according to the manufacturer’s instructions. Cytotoxicity was performed in uninfected VeroE6 cells with same compound dilutions and concurrent with viral replication assay. All assays were performed in biologically independ-ent triplicates.

Results from these studies show that 108600 and 108110 had little or no effect on the viability of Vero E6 cells. In this assay ON123300, a CDK4/ARK5 inhibitor was used as a negative control, which showed no effect on viral replication (Data not shown). Hydroxy-Chloroquine and Remdesivir were used as positive controls and these compounds inhibited the replication of SARS-CoV-2 with an IC₅₀ of 733 nM and 826 nM respectively. In these assays, 108110 inhibited the replication of SARS-CoV-2 with an IC₅₀ of 125 nM and 108600 inhibited replication of SARS-CoV-2 with an IC₅₀ of 13 nM suggesting that 108600 is approximately 63-fold more effective than Remdesivir, the only drug currently used for the treatment of COVID-19 patients. Similarly, 108110 is approximately 7-fold more active than Remdesivir.

### EXAMPLE 2 - ANTI-SARS-COV-2 ACTIVITY OF CELL CYCLE INHIBITORS

Four cell signaling inhibitors were evaluated for anti-viral activity against SARS-CoV2 WA-2020 strain by in vitro infection assay in Vero E6 cells. Infection outcome in the assay was determined by quantitating nucleocapsid (NP) protein in the supernatant of infected cells exposed to the inhibitors by antigen capture ELISA and by measuring infectivity of released virus from the infected cells by plaque assay. The antiviral activity of four cell signaling inhibitors was tested in vitro using SARS-COV2 WA-2020 isolate. All infection assays were performed in Vero E6 cells at Biosafety Level 3 (BSL-3). Two separate assays were conducted to determine the anti-SARS-CoV-2 activity of cell cycle inhibitors. In the first assay higher density of target cells was used (40,000 cells per well) with an MOI (multiplicity of infection) of 0.01 while in the second assay lower cell density was used (2,000 cells per well) with an MOI of 0.05. Both assays were conducted in 96 well assay format. Briefly, Vero E6 cells were plated overnight in poly-L-lysine coated 96 well plate. Cells were treated with the cell signaling inhibitors for 2-3 hours in 200 ml DMEM medium containing 2% FBS at 37° C. in 5% CO2. This step was performed in the BSL-2 laboratory. Plates were then transferred to the BSL-3 laboratory, medium from each well was removed and the cells were infected with appropriate infectious dose of SARS-COV-2 to achieve appropriate MOI (0.01 or 0.05) in the presence of the inhibitors. After incubation for 1 hour at 37° C. in 5% CO2 virus/inhibitor mixture was removed, washed twice with fresh medium, and replenished with fresh medium containing appropriate concentrations of the inhibitors. Plates were then incubated at 37° C. in 5% CO2 for 72 hours. Supernatant was collected and was assayed for NP protein content by antigen capture ELISA (MyBioSource, CA). In addition, plaque assay was performed in the first experiment to determine infectivity of the virus in the supernatant. Control infection included cells and virus treated under similar conditions. For positive inhibition control remdesivir (10, 5, 2.5, 1.25 and 0.625 µM) was used with drug present throughout the infection as defined by its mode of action.

### 6.1 Assay Performed With Higher Density of Target Cells

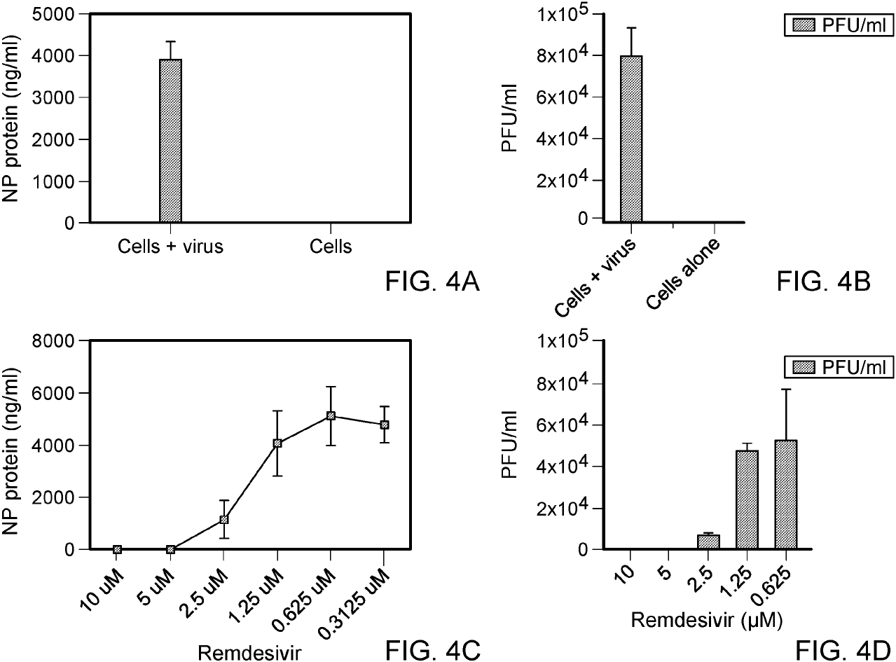

In this experiment Vero E6 cells at high cell density (40,000 cells/well) were used astarget for infection. Infection assay was performed as described above. As shown in FIG. 4A, control infected cells released around 4,000 ng/ml of NP protein into the medium. As expected, cells not exposed to the virus did have no NP protein in the culture. Viruses released from the infected cells were also found to be infectious as evident from the plaque assay with an infectious titer of nearly 80,000 pfu/ml while medium from cells not exposed to virus had no infectious virions (FIG. 4B).

Remdesivir inhibited infection markedly in a dose response manner as evident from the level of NP protein released from the infected cells into the medium (FIG. 4C). This inhibition of infection was also correlated to the recovery of infectious virus released from the infected cells in the presence of remdesivir (FIG. 4D).

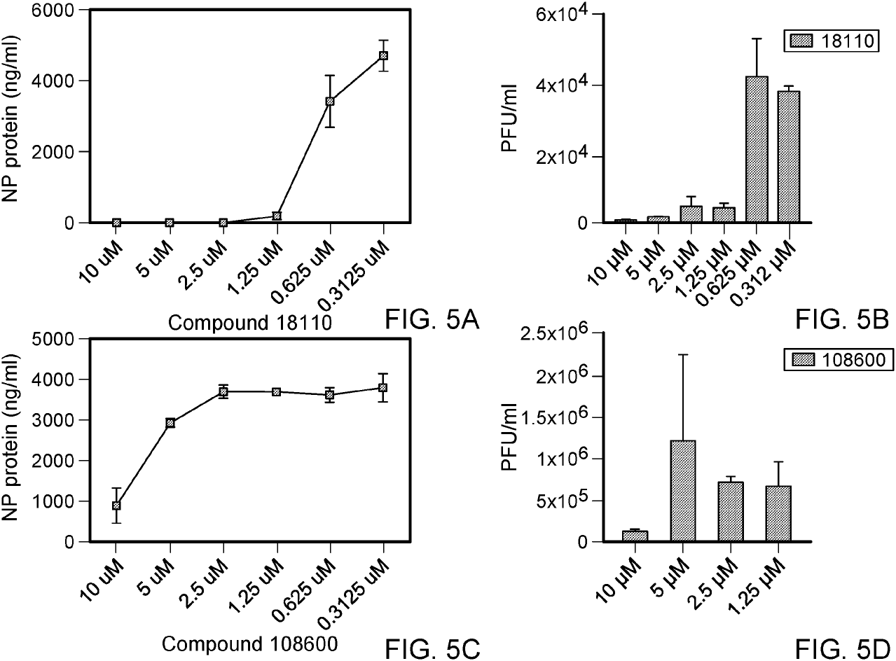

After establishing the positive and virus controls of the assay, anti-SARS-CoV-2 activity of cell cycle inhibitors 108110 and 108600 was evaluated using high density of target Vero E6 cells as done for the control infection. As before, infection output was measured by quantitating NP protein while the infectious titers of the released virus were assayed by plaque assay. As shown in FIGS. 5A and 5C, release of NP protein was inhibited in the presence of both 108110 and 108600 in a dose dependent manner. However, the inhibitor effect was more pronounced with 108110 while for 108600 inhibition was noted only with 10 µM concentration. Moreover, inhibition of release of NP protein for both 108110 and 108600 was correlated to the infectious titers of the supernatant from both inhibitors (FIGS. 5B and 5D).

### 6.2 Assay Performed With Lower Density of Target Cells

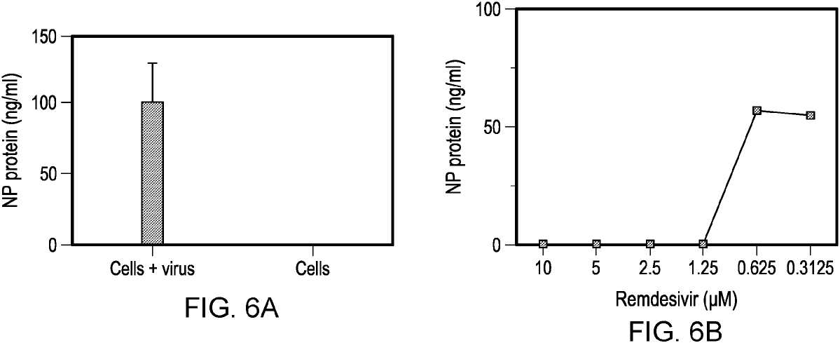

Since the compounds tested here are cell signaling inhibitors and thus affecting cell proliferation, these inhibitors were also tested at a lower cell density of target cells (2000 cells/well) to allow cells to undergo proliferation during the infection. Infection assay was conducted with an MOI of 0.05 as described above. Since NP content in the medium correlated with the infectious titer of the released virus in the previous experiment, another plaque assay of the supernatant to measure infectious titers of the released virions was not performed. As shown in FIG. 6A, lower density of Vero E6 cells infected with SARS-CoV-2 had a lower virus output of around 100 ng/ml of NP protein in the medium. As expected, cells not exposed to the virus had no NP in the culture (FIG. 6A). Similar to the assays performed with high cell density, remdesivir used as a positive control in the assay inhibited infection markedly in a dose response manner as revealed from the level of NP protein released from the infected cells (FIG. 6B).

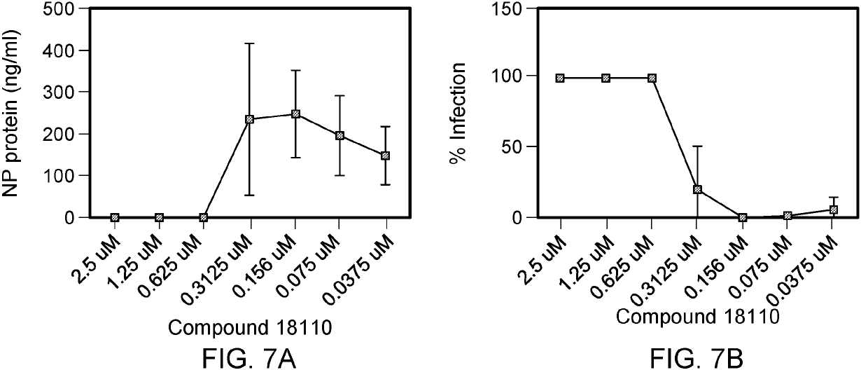

Having established the positive and virus controls in the assay performed with low density of target cells, the anti-SARS-CoV-2 activity of cell cycle inhibitor 108110 using similar conditions was evaluated. No assay was performed with 108600. As shown in FIG. 7, release of virus was inhibited in the presence of this inhibitor in a dose dependent manner. Moreover, the inhibitor showed significant inhibition of infection when compared to the untreated control cells. Results presented above demonstrate that the cell signaling inhibitors 108110 and 108600 inhibited SARS-CoV-2 infection of Vero cells in a dose dependent manner when low density of target cells were used for in vitro infection, and inhibition of infection was also noted with 108110 even at higher density of Vero cells. These assays demonstrate that selection of appropriate density of target cells is an important factor to consider while testing this class of cell signaling inhibitors against SARS-CoV-2 infection in vitro.

The foregoing is not to be limited in scope by the specific embodiments described herein. Indeed, various modifications of the methods provided herein and their equivalents, in addition to those described herein will become apparent to those skilled in the art from the foregoing description and accompanying figures. Such modifications are intended to fall within the scope of the appended claims.

All references cited herein are incorporated herein by reference in their entirety and for all purposes to the same extent as if each individual publication or patent or patent application was specifically and individually indicated to be incorporated by reference in its entirety for all purposes.

What is claimed is:

1. A method for treating an RNA virus infection or a disease associated therewith, comprising administering an effective amount of compound 108600 to a human subject in need thereof.

2. A method for treating an RNA virus infection or a disease associated therewith, comprising administering an effective amount of compound 108110 to a human subject in need thereof.

3. The method of claim 1, wherein the RNA virus is a coronavirus, an influenza virus, a vesicular stomatitis virus (VSV) or a Newcastle disease virus (NDV).

4. The method of claim 2, wherein the RNA virus is a coronavirus, an influenza virus, a vesicular stomatitis virus (VSV) or a Newcastle disease virus (NDV).

5. The method of claim 1 wherein the RNA virus infection is a SARS-CoV-2 infection or COVID-19.

6. The method of claim 2, wherein the RNA virus infection is a SARS-CoV-2 infection or COVID-19.

7. A compound according to Formula (I), or a salt thereof. 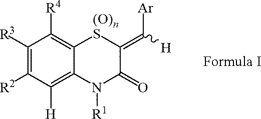
wherein
n is 0, 1, or 2;
R¹ is selected from the group consisting of ―H, ―(C₁-C₆₎alkyl, ―(C₂-C₆)alkenyl, ―C₂-C₆)alkynyl, optionally substituted aryl, optionally substituted heteroaryl, optionally substituted aryl-(C₁-C₆)alkyl, optionally substituted heteroaryl-(Ci-C₆)alkyl, ―C(=O)(C₁-C₆)alkyl, ― C(=O)(C₂-C₆)alkenyl, —C(=O)-optionally substituted aryl, ―C(=O)(CH₂)ₘ-optionally substituted aryl, and ―C(=O)(CH₂)ₚ-optionally substituted heteroaryl;
R², R³, and R⁴ are independently selected from the group consisting of ~~H, halogen, —CN, NR¹⁰R¹¹, —OH, ―OR¹³, ―C₁-C₆ alkoxy, —NO₂, ―(C₁-C₆)alkyl, ―{C₁-C₆,)perfluoroalkyl, ― (C₁-C₆)perfluoroalkoxy, ―C(=O)R¹⁵, ―C(=O)OR¹⁵, ―OC(=O)R¹², ―OC(=O)OR¹², — C(=O)NR¹⁷R¹⁸, —SH, ―S(C₁-C₆)alkyl, ―SR¹³, ―S(=O)R¹³, ―S(=O)₂R¹³, ―OS(=O)₂R¹³, — S(=O)_{q}R¹⁵, ―OS(=O)_{q}R¹⁵, ―S(=O)₂NR¹⁷R¹⁸, ―S(=O)NR¹⁷R¹⁸, optionally substituted aryl, optionally substituted aryl-(C₁-C₆,)alkyl, optionally substituted heteroaryl, optionally substituted heteroaryl-(C₁-C₆,)alkyl, optionally substituted (C₂-C₉)heterocyclyl, optionally substituted (C₂-C₉)heterocyclyl-(C₁-C₆)alkyl, —NH(CH₂)ₘC(═O)OR¹⁴, ―C(=NR¹⁴)NR¹⁴₂, ―C(=N― OR¹⁴)NR¹⁴₂, ―P(=O)(OR¹⁴)₂, and ―OP(=O)(OR¹⁴)₂;
Ar is optionally substituted heteroaryl, optionally substituted (C₁₀-C₁₄)aryl, or 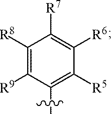
R⁵, R⁶, R⁷, R⁸, and R⁹ are independently selected from the group consisting of —H, —OH, ― OR¹³, —NO₂, halogen, —CN, ―NR¹⁰R¹¹, ―(CH₂)ₘNR¹⁰R¹¹, ―O(CH₂)ₘNR¹⁰R¹¹, —(C₁-C₆)alkyl, ―(CH₂)ₘO(C₁-C₆)alkyl, ―(C₁-C₆)alkoxy, ―(C₁-C₆)perfluoroalkyl, ―(C₁-C₆)perfluoroalkoxy, —SH, ―S(C₁-C₆)alkyl, ―SR¹³, ―S(=O)R¹⁵, ―S(=O)₂R¹⁵, ―C(=O)R¹⁵, ―C(=O)OR¹⁵, ―C(=O)NR¹⁷R¹⁸, ―OC(=O)R¹⁶, ―OC(=O)OR¹², ―OC(=O)NR¹⁷R¹⁸, heterocyclyl, optionally substituted heteroaryl, ―NH(CH₂)ₘC(=O))OR¹⁴, ―OS(=O)₂R¹⁶, ― C(=NR¹⁴)NR¹⁴₂, ―C(═N―OR¹⁴)NR¹⁴₂, ―P(=O)(OR¹⁴)₂, and ―OP(=O)(OR¹⁴)₂;
each R¹⁰ and R¹¹ is independently selected from the group consisting of —H, ―(C₁-C₆)alkyl, — (C₁-C₆)alkoxy, ―C(═O)R¹² ―C(C=O)NR¹⁷R¹⁸, ―C(=O)OR¹², ―C(=NR¹⁴)NR¹⁷R¹⁸, R¹³, optionally substituted aryl, optionally substituted heteroaryl, and ―C(=NR¹⁴)R¹⁵; or R¹⁰ and R¹¹, together with the nitrogen to which they are bound, form an optionally substituted (C₂-C₅)heterocycle;
each R¹² is independently selected from the group consisting of ―(C₁-C₆)alkyl, and optionally substituted aryl;
each R¹³ is independently selected from the group consisting of optionally substituted aryl and -(CH₂)ₘR¹⁶;
each R¹⁴ is independently selected from the group consisting of —H and —(C₁-C₆)alkyl; or two occurrences of R¹⁴ bound to the same nitrogen form a (C₂-C₆)heterocycle, together with the nitrogen atom to which they are bound;
each R¹⁵ is independently selected from the group consisting of —H, ―(C₁-C₆)alkyl, optionally substituted aryl, and NR¹⁴₂;
each R¹⁶ is independently selected from the group consisting of —(C₁-C₆)alkyl, —NR¹⁴₂, and Ar¹;
each R¹⁷ and R¹⁸ is independently selected from the group consisting of —H, —(C₁-C₆)alkyl, — (C₁-C₆)alkoxy, R¹³, optionally substituted aryl, and optionally substituted heteroaryl; or R¹⁷ and R¹⁸, together with the nitrogen to which they are bound, form an optionally substituted (C₂-C₅)heterocycle;
m is independently at each occurrence 1, 2, 3, 4, or 5;
p is independently at each occurrence 0, 1, 2, or 3;
q is independently at each occurrence 0, 1, or 2;
each optionally substituted aryl, optionally substituted (C₁₀-C₁₄)aryl, optionally substituted heteroaryl, optionally substituted aryl-(C₁-C₆)alkyl, optionally substituted heteroaryl-(C₁-C₆)alkyl, optionally substituted (C₂-C₉)heterocyclyl, optionally substituted (C₂-C₉)heterocyclyl-(C₁-C₆)alkyl, and optionally substituted (C₂-C₅)heterocycle is optionally substituted with one or more substituents independently selected from the group consisting of halogen, —CN, —NR¹⁴₂,—(CH₂)ₘNR¹⁴₂, —O(CH₂)ₘNR¹⁴₂, —NR¹⁴C(=O)(C₁-C₆)alkyl, —NR¹⁴C(=O)O(C₁-C₆)alkyl, —NR¹⁴C(=O)NR¹⁴₂, —NR¹⁴C(=NR¹⁴)NR¹⁴₂, —NH(CH₂)ₘC(=O)OR¹⁴, —OH, —NO₂, —(C₁-C₆)alkyl, —(CH₂)ₘO(C₁-C₆)alkyl, —(C₁-C₆)alkoxy, —(C₂-C₆)alkenyl, —(C₂-C₆)alkynyl, — SR¹⁴, —S(=O)R¹⁵, —S(=O)₂R¹⁵, —NR¹⁴S(=O)₂R¹⁵, —(C₁-C₆)perfluoroalkyl, —(C₁-C₆)perfluoroalkoxy, —C(=O)R¹⁴, —C(=O)OR¹⁴, —C(=O)NR¹⁴ ₂, —OC(=O)R¹⁴, — OC(=O)NR¹⁴₂, —OC(═O)O(C₁-C₆)alkyl, —P(=O)(OR¹⁴)₂, —OP(=O)(OR¹⁴)₂, heterocyclyl, and heteroaryl;
Ar¹ is a radical according to Formula II 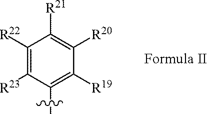
wherein R¹⁹, R²⁰, R²¹, R²², and R²³ are independently selected from the group consisting of —H, —OH, —NO₂, halogen, —CN, —NR¹⁰R¹¹, —(CH₂)ₘNR¹⁰R¹¹, —O(CH₂)ₘNR¹⁰R¹¹, —(C₁-C₆)alkyl, —(CH₂)ₘO(C₁-C₆)alkyl, —(C₁-C₆)alkoxy, —(C₁-C₆)perfluoroalkyl, —(C₁-C₆)perfluoroalkoxy, —SH, —SR¹², —S(=O)R¹⁵, —S(=O)₂R¹⁵, —C(=O)R¹⁵, —C(=O)OR¹⁵, — C(=O)NR17R¹⁸, —OC(=O)OR¹², —OC(=O)NR¹⁷R¹⁸, heterocyclyl, optionally substituted heteroaryl, —NH(CH₂)ₘC(=O)OR¹⁴, —OS(=O)₂R¹⁶, —C(=NR¹⁴)NR¹⁴₂, — C(=N—OR¹⁴)NR¹⁴₂, —P(=O)(OR¹⁴)₂, and —OP(=O)(OR¹⁴)₂; provided that:
i) at least one of R², R³, or R⁴ is other than hydrogen;
ii) when none of R², R³, and R⁴ are —OR¹³, —NHR¹³, —SR¹³, —S(=O)R¹³, or —S(=O)₂R¹³, and Ar is 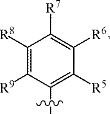
then at least one of R
⁶ and R⁸ is —NO₂ and at least R⁷ is other than hydrogen or halogen; and
iii) when Ar is optionally substituted heteroaryl and none of R², R³, or R⁴ are —OR¹³, —NHR¹³, —SR¹³, —S(=O)R¹³, or —S(=O)₂R¹³, then R¹ is other than hydrogen.

8. The compound of claim 7, wherein n is 0.

9. The compound of claim 7, wherein the wavy bond in the structure of Formula I indicates either (E), (Z), or a mixture of configurations of the double bond to the carbon atom to which Ar and 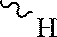
are attached.

10. The compound of claim 7, wherein the double bond in the compound of Formula I is in the Z configuration, n is 0, and Ar is optionally substituted heteroaryl or 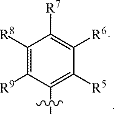
.

11. The compound of claim 7, wherein the optionally substituted heteroaryl group is selected from thiophene-2-yl (thiene-2-yl), thiophene-3-yl (thiene-3-yl), indol-2-yl, indol-3-yl, indol-4-yl, indol-5-yl, indol-6-yl, indol-7-yl, pyrrol-2-yl, pyrrol-3-yl, pyrimidin-4-yl, pyrimidin-5-yl, or pyrimidin-6-yl, any of which can be optionally substituted.

12. The compound of claim 11, wherein the thiophene-2-yl (thiene-2-yl), thiophene-3-yl (thiene-3-yl), indol-2-yl, indol-3-yl, indol-4-yl, indol-5-yl, indol-6-yl, indol-7-yl, pyrrol-2-yl, pyrrol-3-yl, pyrimidin-4-yl, pyrimidin-5-yl, and pyrimidin-6-yl radicals are optionally substituted with a halogen, an alkyl group, such as methyl or ethyl, or an acyl group such as an acetyl group.

13. The compound of claim 12, wherein the acetyl group can be present on the nitrogen of any of the pyrrolyl or indolyl radicals.

14. The compound of claim 12, wherein the pyrimidinyl radicals are substituted with a thioether, or a morpholino group at any of the substitutable position.

15. The compound of claim 7, wherein any of R², R³, or R⁴ can be — S(=O)₂R¹³ wherein R¹³ is —(CH₂)ₘR¹⁶, m is 1, and R¹⁶ is 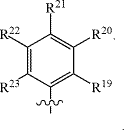
.

16. The compound of claim 7, wherein R² is —S(=O)₂R¹³ wherein R¹³ is — (CH₂)ₘR¹⁶, m is 1, and R¹⁶ is 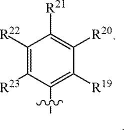
.

17. The compound of claim 7, wherein at least two of R¹⁹, R²⁰, R²¹, R²², and R²³ are halogen atoms and can be the same or different halogen atoms.

18. The compound of claim 17, wherein R¹⁹ and R²³ are both halogen atoms (either the same or different), while R²⁰, R²¹, and R²² are other than halogen.

19. The compound of claim 17, wherein R¹⁹ and R²³ are the same halogen atom, while R²⁰, R²¹, and R²² are other than halogen.

20. The compound of claim 17, wherein R¹⁹ and R²³ are both chlorine atoms and R²⁰, R²¹, and R²² are each hydrogen.

21. A pharmaceutical composition, comprising a compound according to claim 7 and a pharmaceutically acceptable carrier.

22. A method for treating an RNA virus infection or a disease associated therewith, comprising administering an effective amount of a compound according to claim 7 to a human subject in need thereof.

23. The method of claim 22, wherein the RNA virus is a coronavirus, an influenza virus, a vesicular stomatitis virus (VSV) or a Newcastle disease virus (NDV).

24. A method of claim 23, wherein the virus is SARS-CoV-2 or COVID-19.
